(19) United States
(12) Patent Application Publication
(10) Pub. No.: US 2003/0108892 A1
(43) Pub. Date: Jun. 12, 2003

(54) Exciplexes

(76) Inventors: Kenneth T. Douglas, Littleborough (GB); Elena V. bichenkova, Manchester (LV); Ali Sardarian, Manchester (IR)

Correspondence Address: Nixon & Vanderhye, 8th Floor, 1100 North Glebe Road, Arlington, VA, 22201-4714, US

(21) Appl. No.: 10/168,312

(22) PCT Filed: Dec. 20, 2000
(86) PCT No.: PCT/GB00/04898

(30) Foreign Application Priority Data
Dec. 20, 1999 (GB) 9929891.1

(51) Int. Cl. C12Q 1/68; G01N 33/53; C07C 211/57

(52) U.S. Cl. 435/6; 435/7.1; 564/336

(57) ABSTRACT

Compounds capable of forming an intramolecular exciplex on photoirradiation of the compound in water comprise two exciplex forming partners, one being a donor moiety and the other an acceptor moiety, each having at least one aromatic nucleus and being connected by a saturated aliphatic chain having the flexibility to allow said partners to come into exciplex forming relationship. The compounds may be used as labels for oligonucleotides. Certain of the compounds display pH sensitive emission.

The present invention relates to exciplexes, i.e. heterocomplex analogues of excimers.

An excimer is a fluorescent complex formed when two identical complex forming partners (e.g. pyrene) are brought into the correct positional relationship to each other and photoirradiated. An exciplex (the heterocomplex analogue of an excimer) is formed on photoirradiation when (different) donor and acceptor species (e.g. pyrene and dimethyl aniline) come into the correct positional relationship to each other, the exciplex complex then dissociating with emission of fluorescence which is detectably different from that of either of the exciplex forming partners.

Exciplexes have the characteristics that is possible by altering the electron affinity and ionisation potential of the contributing partners to “tune” the emission wavelength of the complex including the wavelength and temporal characteristics, as may be used in time-resolved fluorescence. For example, an exciplex formed from N,N-diethylamine with chrysene emits at ca 420 nm but one formed with N,N-diethylamine with perylene emits at ca 520 nm

More particularly, the emission characteristics can be tuned in a predictable sense by the fine chemical structures of the partners, for example the emitted light frequency being linearly related to the difference in electron donor/electron acceptor strengths of the partners (see for example D. RehM, S. Naturforsch (1970) Vol 25a 1442-1447; J. B. Birks “Photophysics of Aromatic Molecules” published by Wiley Interscience, London.

Solvent polarity is crucial to the behaviour of exciplexes and intermolecular exciplexes do not emit usually in solvents as polar as acetonitrile¹⁻⁵. In contrast, several intramolecular exciplexes exhibit exciplex emission in solvents up to the polarity of acetonitrile⁶⁻¹⁵. This behavioural shift has been ascribed to a change in structure for exciplexes going from compact in nonpolar solvents to loose in polar solvents^{13;14;16}. For strongly interacting exciplex partners, such as aromatic hydrocarbons with dialkylanilines (the most widely studied family, and certainly dominating the literature until very recently), the exciplex arises from a partial charge-transferred state, which is sufficiently stable in nonpolar solvents to fluoresce. Increased solvent polarity preferentially solvates and stabilises charge separation and at a dielectric constant of approximately 14 the pyrene:diethylaniline pair has an exciplex absorption spectrum identical with the ion pair, pyrene⁻: PbNF-t₂⁺. Fluorescence quantum yields and lifetimes of exciplexes usually decrease with solvent polarity^{3-5;11} the former more sensitively, but effects on lifetimes are more variable.

Intramolecular exciplexes have recently been discovered which emit in solvents as polar as DMSO, or even 20% aqueous CH₃CN¹⁷ Intermolecular exciplex luminescence in polar: nonpolar solvent mixtures (such as DMSO-benzene 18 or water-THF or water-dioxane¹⁹ can be enhanced by magnetic fields. In none of these situations was use of a fully aqueous medium possible to observe exciplex emission even with the enhancement of magnetic field application. Literature data indicate that increasing water percentage in mixed solvents suppresses exciplex formation^{12;17.}

Emission of exciplexes in fully aqueous media is rarer and usually only occurs under special circumstances, for example in the special environments provided by some biomacromolecules, or in the presence of additives (such as cyclodextrins^{20;21}, cyclophanes²², polyanions²³). Exciplexes in water can be stabilised by polyanions (especially poly(vinyl sulphate), but also chondroitin sulfate C and heparin). The exciplex formed from excited 3,3′-diethylthiacyanine iodide (THIA) and cool acridine orange (AO) in the presence of chondroitin sulphate and other polyanions including DNA (emitting at 560 nm) does not form in their absence and is destroyed by an increase in percent ethanol as co-solvent²³. The enhancement of THIA fluorescence by the matrix depends also on structure of the polyanionic matrix (chondroitin sulphate C is about 4 times as effective chondroitin sulphate A). DNA enhances THIA fluorescence twice as strongly as chondroitin sulfate C (data quoted but unpublished). Heparin also facilitates exciplex formation in the aqueous environment of THIA bound to a polymer. The synthetic polymer poly (vinylsulphate) quenches the monomer fluorescence of AO completely, but facilitates exciplex emission between THIA and AO (λₑₘᵢₜ=560 nm). The authors provide arguments that the emissions detected are indeed exciplexes and not heteroaggregates of THIA and AO. In the absence of such additives, aqueous exciplex emission, if it exists, is too weak to have been detected to date.

Inorganic exciplexes behave differently from exciplexes formed from neutral organic molecules. For the Ru(bpy)₃²⁺ and Ru(phen)₃²⁺/Ag⁺ systems exciplex emission spectra are much less red-shifted with increased [CH₃CN] in H₂O: CH₃CN mixtures relative to the spectrum in H₂O. This study showed that exciplex stabilisation by solvent polarity has the opposite tendency for neutral molecules compared to the exciplex from two ionic species of the same sign^{24;25}. In spite of the enormous effect that addition of MeCN to H₂O has on the overall properties of the RuL₃²⁺/Ag⁺ exciplex system, these compositional changes have a remarkably small effect on the formation constants for the Ag⁺ exciplexes (even though solvent changes affect photo-properties)²⁴.

The use of applied, magnetic. fields. has permitted exciplex emission to be studied in water-solvent mixtures such as water-dioxan up to 20% water v/v. For a series of intramolecular exciplexes, exciplex fluorescence increased steeply as the magnetic field went from 0 to 1.0 Tesla (and then decreased gradually to 9 T (similarly the mean lifetimes)¹⁸. The exciplex luminescence increase found with increased magnetic fields is dependent on: (a) the particular exciplex partners, (b) the particular solvent mixtures (the increase is maximal at γ=15 for THF: MeOH and ˜17 for dioxan: H₂O), (c) the magnetic field strength (plateau at approx 150 Gauss for 520 nm emission of pyrene: DMA and (d) the ionic strength¹⁹.

Exciplexes of undefined structures have been reported for proteins and (oligo)nucleotides and nucleic acids with a number of ligands, which interact with some part(s) of the protein (such as tryptophan residues) or the nucleic acid/nucleotide (usually base or base-pair(s)). In view of the complexity of these systems and the lack of precision in structural definition currently available for the contributors to the exciplex partners, such systems have not permitted the impact of a fully aqueous environment on exciplex properties to be explored.

There is no report of intramolecular organic exciplex emission in fully aqueous media, and consequently no information of the influence on exciplex properties of media parameters, including ionic strength, temperature, pH, co-solvents, buffer materials, metal ions, detergents-etc.

Cox et al¹¹⁸ have observed a pH-dependent increase in an exciplex signal following an ionisation profile corresponding to a pH value of 8.9. This discovery had the following limitations: (1) it only occurred for 1-α-naphthyl-3-(dimethylamino)propane in the presence of high concentrations (0.01M) of β-cyclodextrin and the pH sensitivity was such that the maximal change in signal occurred between pH 8 and 10, outside the physiological range; (2) the binding strength of the complex formed between 1-α-naphthyl-3-(dimethylamino)propane and β-cyclodextrin, necessary for exciplex signal to be detectable, was weak and thus extremely high concentrations of β-cyclodextrin would have to be added (this would limit use in living. tissue in view of the. properties and toxicities of β-cyclodextrin).

According to a first aspect of the present invention there is provided a compound capable of forming an intramolecular exciplex on photoirradiation of the compound in water, said compound comprising two exciplex forming partners each having at least one aromatic nucleus and being connected by a saturated aliphatic chain having the flexibility to allow said partners to come into exciplex forming relationship.

The present invention thus provides the significant advance of compounds which are capable of forming intramolecular exciplexes in water. Such exciplex formation may be achieved in systems which are, in effect, 100% aqueous but formation is not limited to such systems and may also be achieved in liquids which are partly aqueous and also in non-aqueous medium polarity liquids (e.g. THF) and low polarity liquids (e.g. toluene). The exciplex emission is achieved without the need for additives such as cyclodextrin or polyanionic compounds and without the requirement for application of the magnetic field.

The compounds of the invention allow the use of aqueous media to exploit properties of exciplexes that were previously restricted to non- or low-percentage aqueous environments such as lasers, dyes to act as fluorescent or circular dichroism detectors when the intra molecular exciplexes have been used to label other materials including proteins, nucleic acids and (oligo)nucleotides and their analogues such as PNAs, glycoproteins, solid materials including ceramics, transistors, semiconductors, insulators and glasses.

Moreover, as detailed more fully below, at least certain of the compounds in accordance with the invention provide pH sensitive exciplex emission in aqueous systems. Thus for example, there may be no exciplex emission below a certain pH but emission above that pH. Alternatively or additionally the emission characteristics may vary continually over a pH range. Thus for the compounds which demonstrate pH sensitive emission may be used as the basis for a molecular pH trigger or switch. More particularly, and by way of example only, certain compounds in accordance with the invention (e.g. SM-2—see Example 1 infra) have sensitivity to fluorescence of the exciplex at around pH 7.5 and may be used as the basis for analytical methods for in vitro systems or cell based systems. At low pH (e.g. pH 6) there is no detectable signal but changing the pH to 7.5 or higher gives exciplex emission, this the compound (or an analytical device based on the compound) could be used, without the need for additives such as cyclodextrins as was necessary in the work of Cox et al, to:

(a) register pH changes in an environment such as a living or fixed cell, or in a curvette or other container including cell sorter devices, and be used in assays to other determinations, or

(b) induce a signal by manipulation of the pH of the environment externally, such as by added acid, base or other change in conditions which leads to a pH change.

Thus according to a second aspect of the present invention there is provided a method of detection wherein there is used, as a label, a compound in accordance with the invention and said method comprises irradiating the sample under test with electromagnetic radiation capable of providing exciplex formation in said label (if present) and detecting for exciplex formation.

The sample which is irradiated preferable has an alkaline pH. The method may involve increasing the pH of the sample (e.g. from neutral to alkaline) to achieve exciplex formation.

The compounds in accordance with the invention comprise two exciplex forming partners each having at least one aromatic nucleus and being connected by a saturated aliphatic chain having a length and flexibility to allow the partners to come into exciplex forming relationship on irradiation with light of the appropriate wavelength. By the term “saturated aliphatic chain” we mean that “line” of atoms which links the two exciplex forming partners does not include an atom in that “line” which has move than one bond to another single atom. Thus, for example the carbon atom of a carbonyl group is regarded as being an unsaturated atom. The presence of unsaturated atoms in the aliphatic linker chain will restrict rotation (about the unsaturated bond) and act to prevent the (potential) exciplex partners coming into exciplex forming relationship.

It should be noted that the saturated aliphatic linker chain may itself be substituted (possibly with groups containing unsaturated atoms provided that such atoms are not in the “line” of atoms connecting the two exciplex forming partners), such substituted compounds are also to be regarded as being within the scope of the invention, the substitution may for example be with a functional group (e.g. an amino group) which permits covalent attachment of the compound (as a label) to a molecule to or other entity to be labelled, e.g. a nucleic acid, (oligo)nucleotide or protein. Such labelled entities are also to be regarded as being within he scope of the invention.

Preferably the saturated aliphatic linker comprises 2 to 9 (e.g. 2 to 6) saturated atoms as the rink between the two exciplex-forming partners, More preferably the linker comprises 2 to 4, and ideally 3, saturated atoms as the link. The saturated atoms if the linker may be carbon atoms (e.g. provided by methylene groups) or may be comprised partly or wholly of other atoms, e.g. nitrogen, sulfur or oxygen

Preferred examples of linker include —CH₂—CH₂—, CH₂—CH₂—CH₂, and —CH₂—NH—CH₂— in which the free terminal bonds are bonded to the exciplex forming partners.

The exciplex forming partners in compounds of the invention will comprise donor and acceptor moieties (each incorporating at least one aromatic nucleus) which may be selected (by way of example only) from residues of any of the following compounds, namely

(i) benzene, naphthalene, anthracene, phenanthrene, pyrene and crysene, phenanthrene;

(ii) derivatives of residues of compounds identified under (i), particularly (but not exclusively) amino or substituted amino derivatives such as N-alkylamino substituted derivatives (where the alkyl groups preferably have 1 or 2 carbon atoms); and N,N-dialkyl substituted derivatives where the two alkyl groups may be but need not necessarily be identical to each other

(iii) phthalimide or substituted derivatives thereof

For the residues identified under (i) and (ii), the saturated aliphatic linker will preferably be bonded directly to the carbon atom of the aromatic ring. In the case of (iii) the linker will be bonded to the imide ring which (because the phthalimide residue is the exciplex partner) does not constitute part of the saturated aliphatic linker.

It is particularly preferred in accordance with the invention that the donor exciplex partner is a 1-pyrenyl group and the acceptor is an amino, N-allylamino or N,N-dialkylamino substituted benzene or naphthalene nucleus. Preferably the amino (or alkyl substituted amino group) is of the 4-position relative to the position at which the linker is bonded to the-benzene or naphthalene. The alkyl groups may be methyl or ethyl groups. For an N,N-dialkylamino compound the alkyl group may be the same or different.

The invention will be illustrated by the following non-linking Examples which are studies based on the compounds SM1-8 for which the structures are shown in Scheme 1. The synthesis of compounds SM1-8 is described in the Appendix A. Compounds SM-2, SM-3 and SM-8 are in accordance with the invention. The other compounds are comparative.

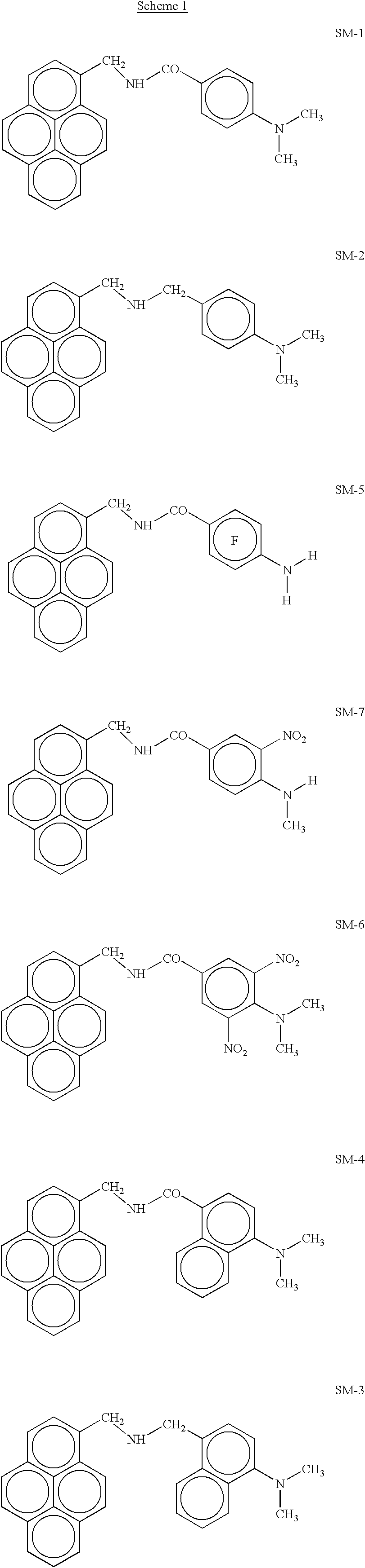

### 1. EXAMPLE 1

### Study of SM-2

1.1 Exciplex Formation by SM-2 in Non-Polar and Medium Polarity Solvents.

The fluorescence characteristics of SM-2 (a compound in accordance with the invention) in a variety of non-polar and medium polarity solvents were studied (using the procedures described more fully below) and the results are set out in Table 1, and shown in Figures of the accompanying drawings.

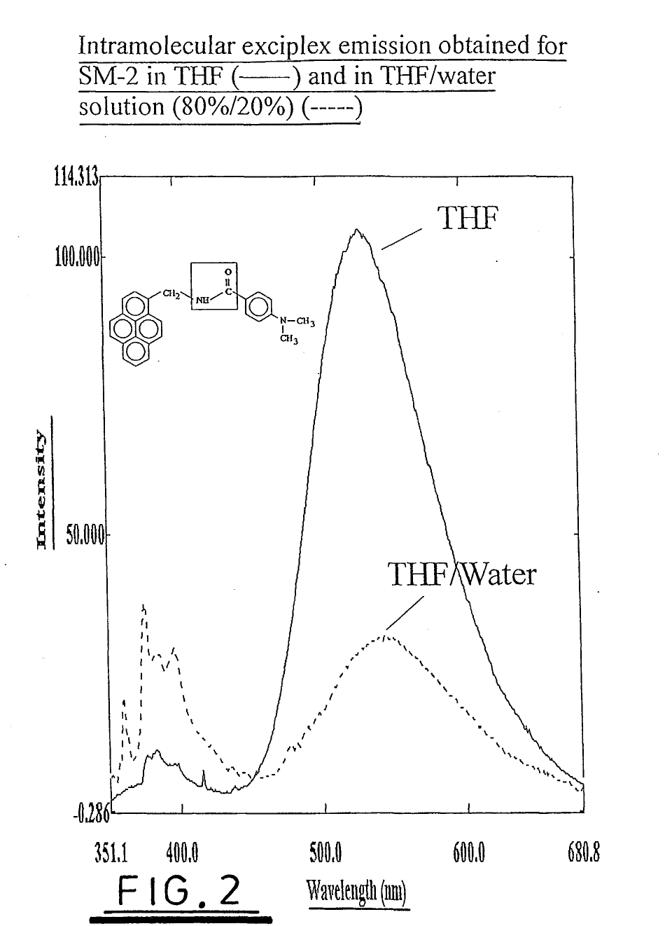

FIG. 1 represents the emission spectra of 1-methylamino-pyrene (solid curve) and SM-2 (dashed curve (ii)) in toluene (the dielectric constant is 2.34²⁶) at 10⁻⁵ M concentration (excitation wavelength was 342 nm). The λₘₐₓ for monomer emission was 378 nm for both 1-methylamino-pyrene and for SM-2. An additional emission band with λₘₐₓ 489 nm was observed for SM-2. This was attributed to intramolecular exciplex formation between the Pyr (pyrene) and DMA (dimethyl aniline) moieties of SM-2. This new emission band could not be attributed to intermolecular excimer formation between the Pyr moieties of two molecules of SM-2 because no excimer formation was discovered for 1-methylamino-pyrene in a control experiment performed at the same concentration and under identical conditions (FIG. 1, solid curve). It should be emphasised that addition of dimethylaniline (10⁻⁵ M final concentration) to the toluene solution of 1-methylamino-pyrene (10⁻⁵ M) in a separate control experiment did not result in intermolecular exciplex formation. In fact, intermolecular exciplex formation started to be observable only after the addition of a 100-fold excess of dimethylaniline (10⁻³ M final concentration) to the toluene solution of 1-methylamino-pyrene (10⁻⁵ M) (data not shown).

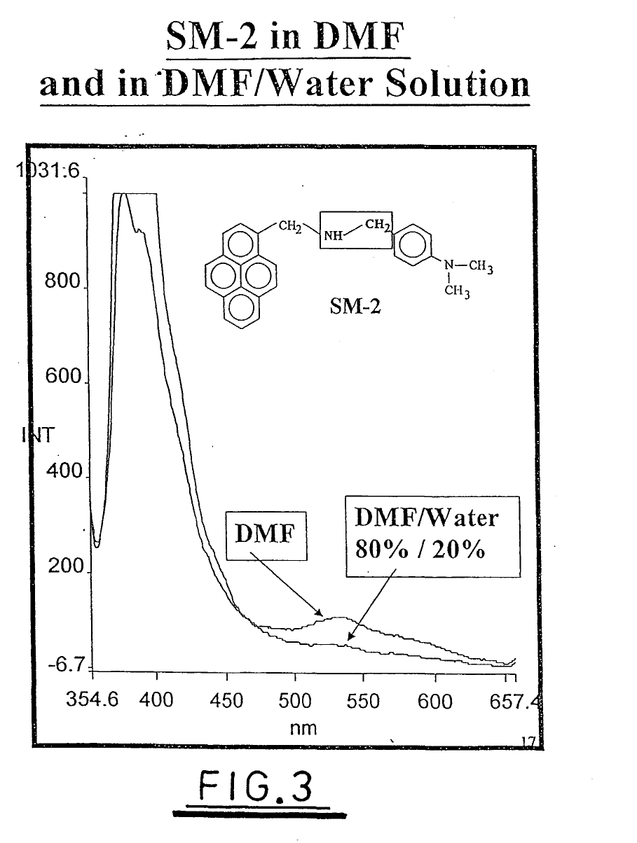

SM-2 was also found to form an intramolecular exciplex in 100% THF (FIG. 2, solid curve) with emission λₘₐₓ 522 nm and excitation wavelength of 342 nm (the dielectric constant for THF is 7.58²⁷). It was found that the λₘₐₓ of the exciplex emission depends strongly on solvent polarity (Table 1). Exciplex emission was also detectable in aqueous-THF solution (H₂O:THF (2:8)). However, the presence of water decreased significantly the ability of SM-2 to form the exciplex based on relative intensity (FIG. 2, dashed curve). In fact, in 100% TBF the I_{EX}: I_{M} ratio was 0.9:0.1 while in H₂O:THF (2:8) mixture the I_{EX}:I_{M} ratio was inverted to 0.65:0.35, where I_{EX} and I_{M} are the relative integral intensities of exciplex and monomer emission, respectively (Table 1).

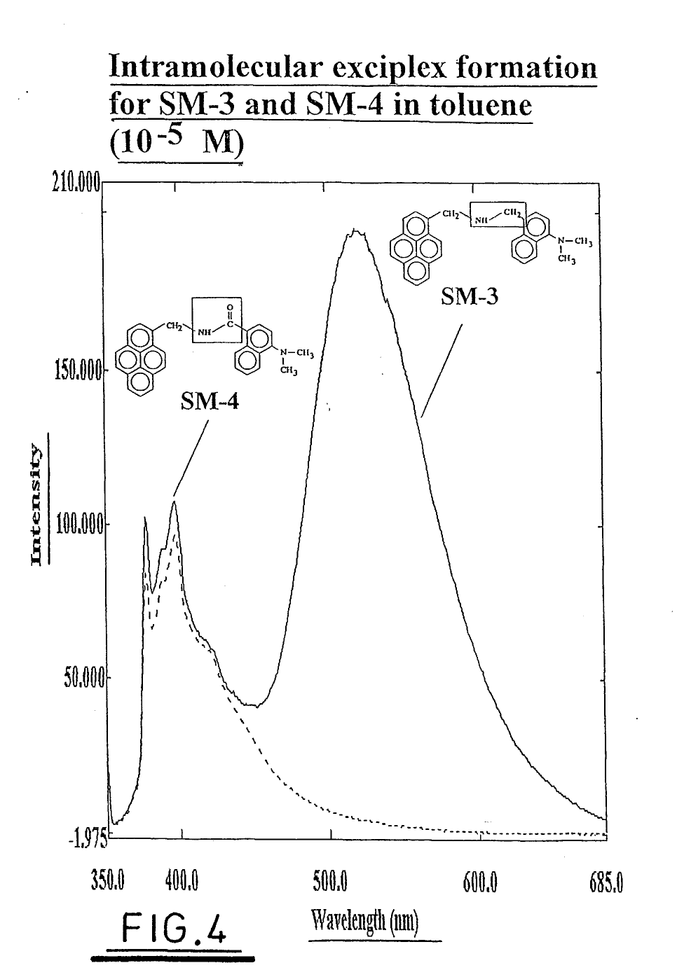

Relatively weak intramolecular exciplex formation for SM-2 was found to occur in medium polarity solvents, e.g. in DMF (the dielectric constant is 36.7²⁷). (FIG. 3, Table 1). However, the addition of H₂O to this DMF solution (to give H₂O:DMF (2:8), which has a net dielectric constant of 45.07 at 20° C.) almost completely suppressed exciplex formation (FIG. 3). This is in accord with literature reports of the effects of increasing water percentage in mixed solvent systems^{12;17}

No exciplex emission was observed for SM-2 in acetonitlile (dielectric constan

t 35.9²⁷), or in acetonitrile/water solutions (Table 1).

TABLE 1
Fluorescence characteristics of monomer and exciplex emission
obtained for SM-2 in different solvents and solvent mixtures.
I_{EX} and I_{M} values cannot
be compared between solvent systems in this table.

| | | Dielectric | | | | |
|---|---|---|---|---|---|---|
| | Refractive | constant, | λₘₐₓ (nm) | λₘₐₓ (nm) | | |
| Solvent | index, η | ∈ (debye) | monomer | exciplex | I_{M}^{d} | I_{EX}^{e} |
| Toluene | 1.4893 | 2.34^{a} | 378 | 489 | 0.31 | 0.69 |
| THF | 1.4040 | 7.58^{b} | 378 | 522 | 0.10 | 0.90 |
| THF/H₂O | 1.3892 | 21.77^{c} | 374 | 522 | 0.35 | 0.65 |
| (80%/ | | | | | | |
| 20%) | | | | | | |
| DMF | 1.4270 | 36.7^{b} | 378 | 535 | 0.86 | 0.14 |
| DMF/H₂O | 1.4083 | 47.05^{c} | 378 | 535 | 0.99 | 0.01 |
| (80%/ | | | | | | |
| 20%) | | | | | | |
| Aceto- | 1.34576 | 35.9^{b} | 378 | — | 1.00 | — |
| nitrile | | | | | | |

^{a}Dielectric constant was taken from ²⁶;
^{b}Dielectric constant was taken from ²⁷;
^{c}Dielectric constant was calculated from the equation: ∈* = A∈_{M} + B∈_{N}, where ∈₁ and ∈₂ - are the respective dielectric constant of the solvents M and N, and where A and B are is a proportions of the solvent M and N, respectively. For refractive indices of mixed media a similar equation was used.
^{d}Normalised fractional intensity of the monomer emission, relative to exciplex in the same experiment ( sum of intensity for monomer plus exciplex = 1.0)
^{e}Normalised integral intensity of the exciplex emission, relative to monomer emission.

1.2 Exciplex Formation by SM-2 in Aqueous Solutions

The fluorescence characteristic of SM-2 in aqueous solutions (the dielectric constant for water is 78.54²⁶) was tested and the results are set out in Table 2 and shown in Figures of the accompanying drawings.

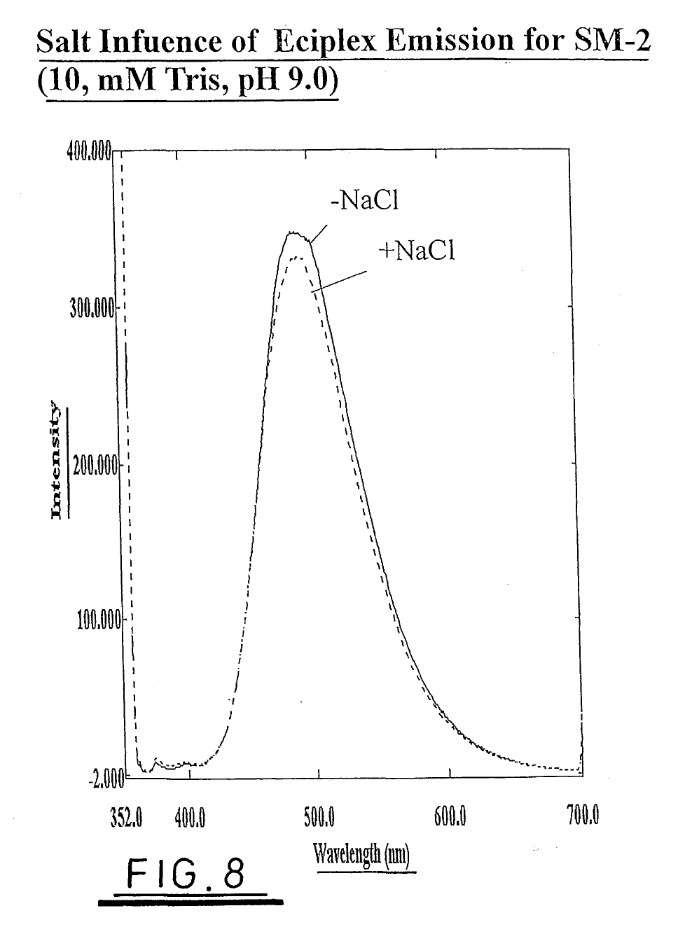

FIG. 7 represents the emission spectra of 1-methylamino-pyrene (dashed curve) and SM-2 (solid curve) in 0.01 M Tris buffer (pH 9.0) prepared in H₂O, at 10⁻⁵ M concentration (excitation wavelength was 350 nm). The λₘₐₓ for monomer emission was 378 nm for both 1-methylamino-pyrene and SM-2. An additional intense emission band with λₘₐₓ of 484 nm was observed for SM-2, which is attributed to intramolecular exciplex formation between Pyr and DMA moieties of SM-2. This new emission band is unlikely to arise from intermolecular excimer formation between the Pyr moieties of SM-2 because no excimer formation was discovered for 1-methylamino-pyrene in a control experiment performed at the same concentration and under identical conditions (FIG. 7). This is the first reported case of intramolecular exciplex formation in aqueous solution that has not required an additive, such as cyclodextrins²⁰ or polyanions, such as chondroitins^{23.}

At a pH 6, no emission from SM-2 was observed (see Table 2).

TABLE 2
Fluorescence characteristics of monomer and exciplex emission
obtained for SM-2 in water.

| | | Dielectric | | | | |
|---|---|---|---|---|---|---|
| | Refractive | constant, | λₘₐₓ (nm) | λₘₐₓ (nm) | | |
| Solvent | index, η | ∈ (debye) | monomer | exciplex | I_{M}^{d} | I_{EX}^{e} |
| 10 mM, | 1.3330 | 78.54^{a} | 378 | 484 | 0.05 | 0.95 |
| Tris | (water) | | | | | |
| buffer | | | | | | |
| (pH 9.0) | | | | | | |
| 10 mM, | 1.3330 | 78.54^{a} | 378 | — | 1.00 | 0 |
| Tris | (water) | | | | | |
| buffer | | | | | | |
| (pH 6) | | | | | | |

^{a}Dielectric constant was taken from ²⁶;
^{d}Normalised fractional intensity of the monomer emission, relative to exciplex in the same experiment (sum of intensity for monomer plus exciplex = 1.0)
^{e}Normalised integral intensity of the exciplex emission, relative to monomer emission.

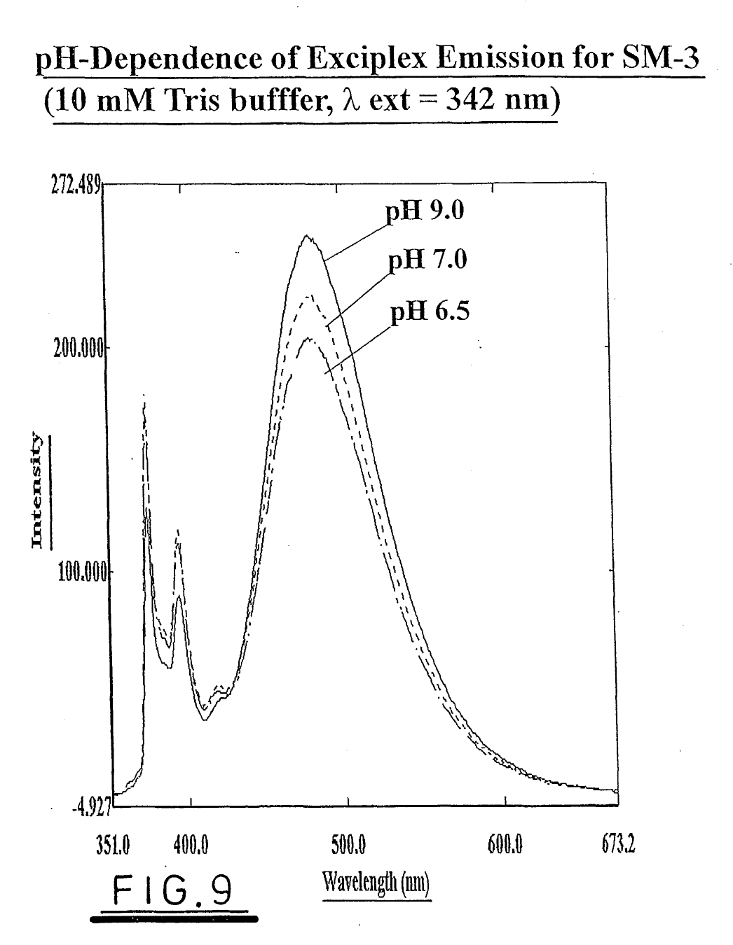

We also carried out a preliminary investigation of the influence of salt concentration on exciplex formation in aqueous solvents, close to physiological conditions. FIG. 8 shows emission spectra of SM-2 in 0.01M Tris buffer (pH 9.0) in the absence (solid curve) and in the presence (dashed curve) of 0.1M NaCl. (A 5% decrease of the fluorescence intensity after the addition of 100 μl of 2M NaCl into the 2 ml cuvette can be attributed to the decrease in concentration of SM-2). FIG. 8 shows that the presence of NaCl slightly quenched exciplex emission. The decrease in the intensity of exciplex emission was found to be 10% (after concentration correction). However, under approximately physiological conditions (0.1M NaCl, 0.01M sodium phosphate buffer, pH 9.0) intramolecular exciplex formation is still easily detectable for system SM-2.

1.3 Effect of pH on Exciplex Formation by SM-2 in Aqueous Solutions

The effect of pH on the fluorescence characteristics of SM-2 in aqueous solution (dialectic constant for water in 78.54²⁶) was tested and the results are set out in Table 3 and shown in Figures of the accompanying drawings.

The pH-dependence of exciplex formation in aqueous solutions was analysed in a preliminary scouting study for SM-2 (10⁻⁵ M) in Tris buffer (0.01 M) with a pH range of 5.5-12.7. The data on pH-dependence of the normalised intensity of monomer and exciplex emission are presented in Table 3.

TABLE 3
pH-dependence of normalised fluorescence intensities of monomer
and exciplex emissions for SM-2 (10⁻⁵ M) in 10 mM Tris buffer.

| | I_{M}^{a} | I_{EX}^{b} |
|---|---|---|
| pH | 378 nm | 484 nm |
| 11.3 | 0.50 | 0.50 |
| 10.12 | 0.56 | 0.44 |
| 9.55 | 0.54 | 0.46 |
| 9.18 | 0.55 | 0.45 |
| 8.96 | 0.60 | 0.40 |
| 8.44 | 0.59 | 0.41 |
| 8.12 | 0.61 | 0.39 |
| 7.72 | 0.71 | 0.29 |
| 7.45 | 0.75 | 0.25 |
| 7.20 | 0.87 | 0.12 |
| 6.99 | 0.98 | 0.02 |
| 6.34 | 1.00 | 0 |
| 6.00 | 1.00 | 0 |

^{a}Normalised intensity of monomer emission at 378 nm; normalised such that the sum of monomer and exciplex emission intensities for any given pH equal 1.00
^{b}Normalised intensity of exciplex emission at 484 nm;

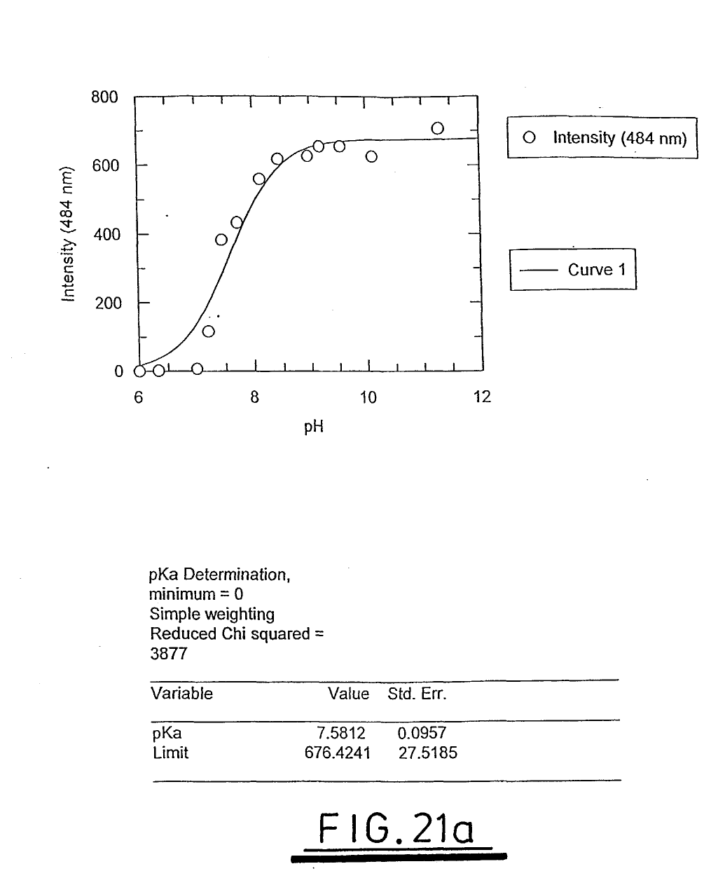

The series of emission spectra of SM-2 versus pH is shown in FIG. 21 (λ_{excitation}, 350 or 340 nm). In acidic and neutral media (pH≦7.0) SM-2 exciplex emission is not detected in aqueous solution and this is consistent with the known prior art on exciplexes in polar media. Surprisingly, increase of pH above 7.0 results in exciplex emission. Moreover, the normalised intensity of exciplex emission I_{EX} increases with the increase in pH, reaching a plateau at around pH 9.0. A plot of relative exciplex emission intensity versus pH (FIG. 21a) is overlaid in this Figure by a theoretical ionisation curve corresponding to a single acid ionisation of pKₐ 7.58±0.10.

1.4 Effect of Some Other Component of the Medium

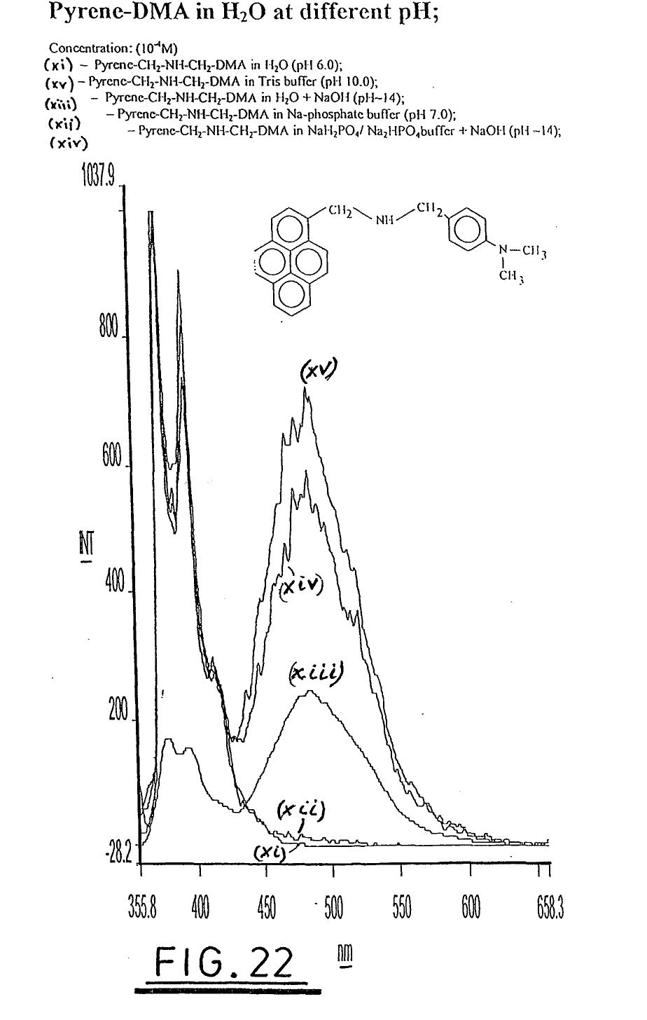

FIG. 22 gives a pictorial representation of the importance of alkaline pH for exciplex emission. In pure water (pH 6.0, curve (xi)) and in 0.01M sodium phosphate buffer (pH 7.0, curve (xii)) SM-2 presents only monomer emission, λₘₐₓ 378 nm. The addition of a drop of concentrated NaOH into each of the above solutions (final pH˜14) resulted in the appearance of intense exciplex emission (curves (xiii) and (xiv) corresponding to water and to buffer systems, respectively). The curve (xv) corresponding to the emission spectrum of SM-2 in Tris buffer (pH 10.0) is shown for comparison. These experiments clearly show that exciplex emission is strongly affected by the pH of the solution, but that it is not very sensitive to buffer composition. Based on the above data, it is reasonable to propose that exciplex formation in aqueous solvents is strongly influenced by the extent of —N(CH₃)₂ protonation, namely, by the pKₐ of the —N(CH₃)₂ group, or possibly by the protonation properties of the amino group in the linker itself. Possibly, the protonation of nitrogen atom of the —N(CH₃)₂ group and/or —NH-linker site completely suppresses the charge-transfer between DMA (donor) and Pyr (acceptor).

1.5 Exciplex Formation in Aqueous Solutions in the Presence of P-cyclodextrin

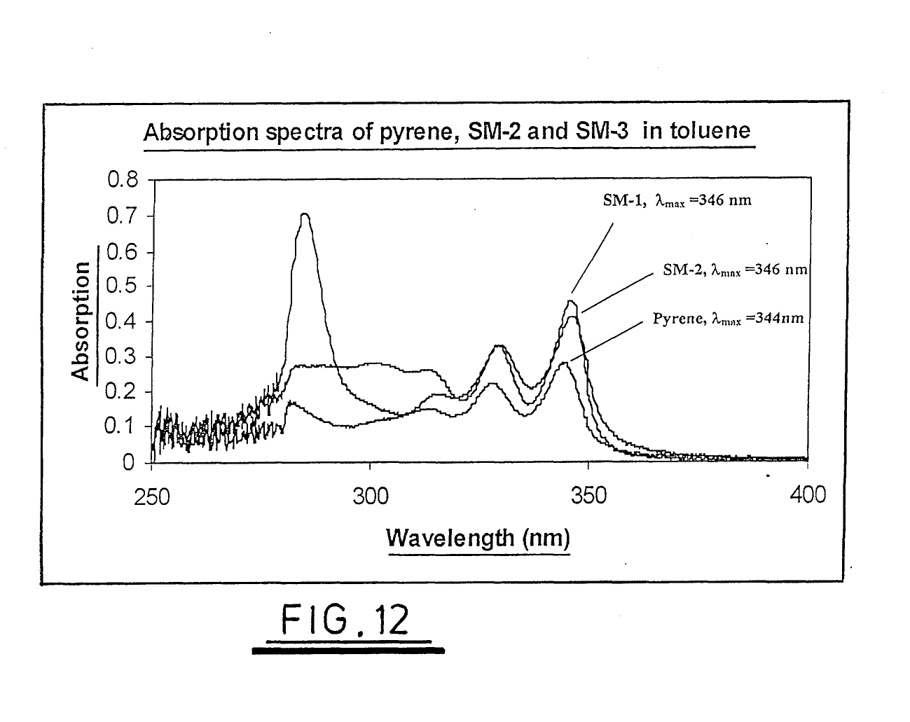

β-Cyclodextrin is known to form hydrophobic cavities in which intramolecular exciplexes can be formed by some small molecules in polar solvents (even in aqueous solutions)^{20;21}. Thus, we studied the possibility of exciplex formation by SM-2 in aqueous solutions in the presence of β-cyclodextrin. This test experiment was performed in 0.01 M Tris buffer (pH 10.0), using conditions (concentration of SM-2=10⁴ M) which were shown earlier to be suitable for exciplex formation (FIG. 11, curve (i). Surprisingly, the addition of β-cyclodextrin (final cuvette concentration 0.01M) completely suppressed exciplex formation (FIG. 11, curves (ii) and (iii)). This can be explained by the large dimensions of the Pyr acceptor component of SM-2 preventing the insertion of Pyr into the β-cyclodextrin cavity; the small DMA moiety may be located inside the β-cyclodextrin.

### 2. EXAMPLE 2

### Study of SM-3

2.1 Exciplex Formation by SM-3 in Non-Polar and Medium Polarity Solvents

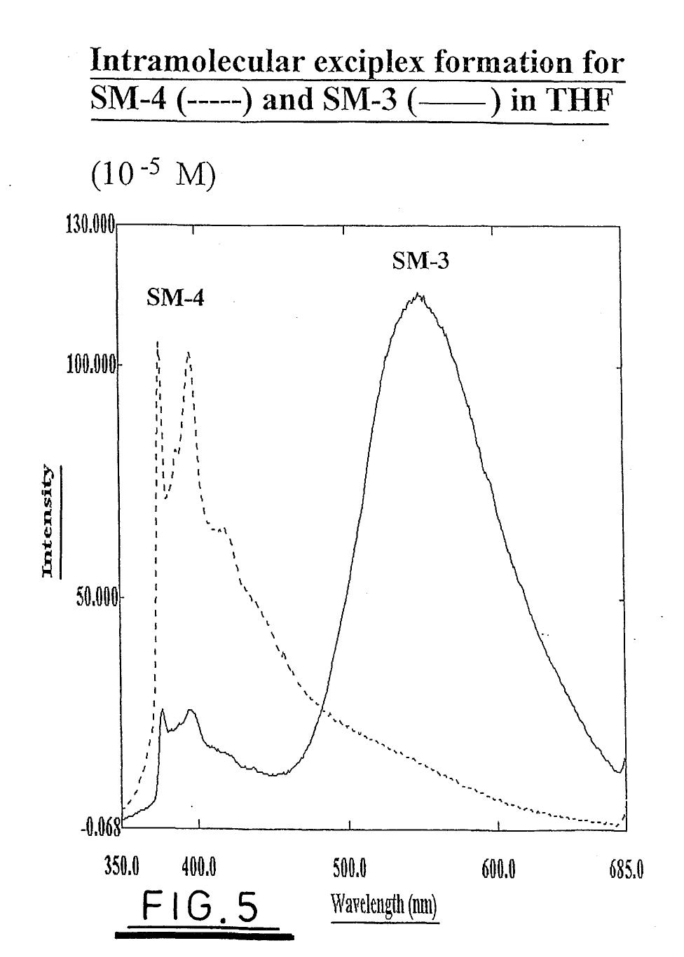

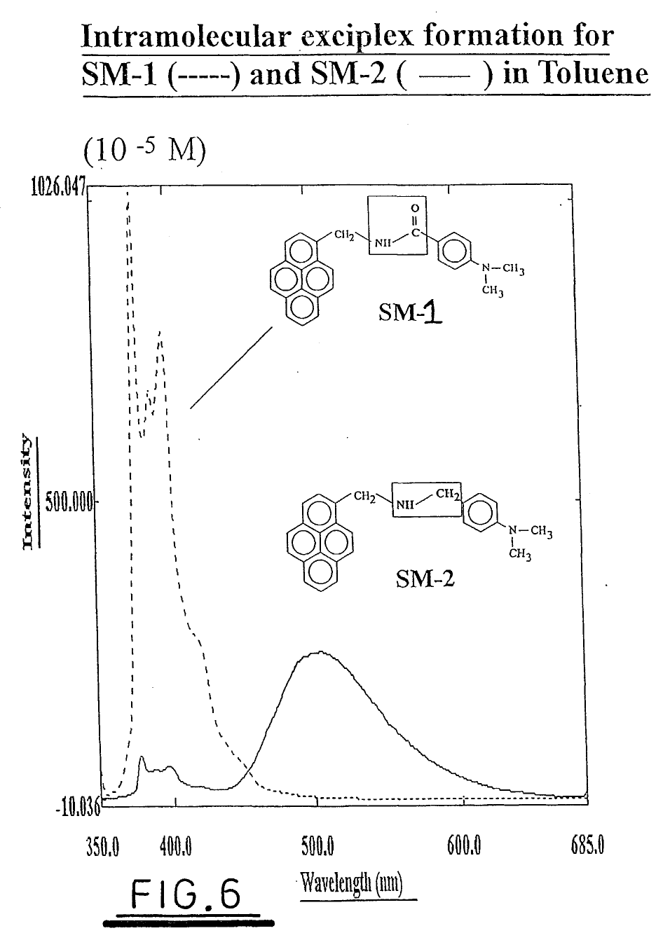

SM-3, which contains the pyrene group as an acceptor and DMN (N,N-dimethyl naphthalyl) group as a donor, was found to form an intramolecular exciplex in toluene or acetonitrile media. FIGS. 4 and 5 represent the emission spectra of 10⁻⁵ M SM-3 and SM-4 solid and dashed curves, respectively) (blue) (curve (ix)) in toluene and in THF respectively (excitation wavelength was 344 nm). The value of λₘₐₓ for monomer emission of the SM-3 was 383 nm in both toluene and THF, while λₘₐₓ for exciplex emission was 515 nm in toluene and 530 nm in THF (Table 4). As seen previously for SM-2, increase of solvent polarity results in a red shift of the exciplex λₘₐₓ. Additionally, the λₘₐₓ for exciplex emission for SM-3 was red-shifted, compared with the SM-2 under similar conditions. The proportion of fluorescence emission arising from exciplex relative to monomer for SM-3 is slightly less than for SM-2, both in toluene and THF.

TABLE 4
Fluorescence characteristics of monomer and exciplex emission obtained
for SM-3 in different solvents.
I_{EX} and I_{M} values cannot be compared between solvent
systems in this table.

| | Dielectric | λₘₐₓ (nm) | λₘₐₓ (nm) | | |
|---|---|---|---|---|---|
| Solvent | constant (debye) | monomer | exciplex | I_{M}^{c} | I_{EX}^{d} |
| Toluene | 2.34^{a} | 383 | 515 | 0.48 | 0.58 |
| THF | 7.58^{b} | 383 | 530 | 0.57 | 0.43 |
| Tris pH 10 | 78.54^{a} | 377 | 482 | 0.05 | 0.95 |
| pH 6.34 tris | 78.54^{a} | 377 | 482 | 0.11 | 0.89 |

^{a}Dielectric constant was taken from ²⁶;
^{b}Dielectric constant was taken from ²⁷;
^{c}Normalised integral intensity of the monomer emission, relative to exciplex
^{d}Normalised integral intensity of the exciplex emission, relative to monomer

2.2 Exciplex Formation by SM-3 in Aqueous Solutions

The fluorescence characteristics of SM-3 in aqueous solutions (the dielectric constant for water is 78.54²⁶) was tested and the results are set out in Table 5 and shown in Figures of the accompanying drawings.

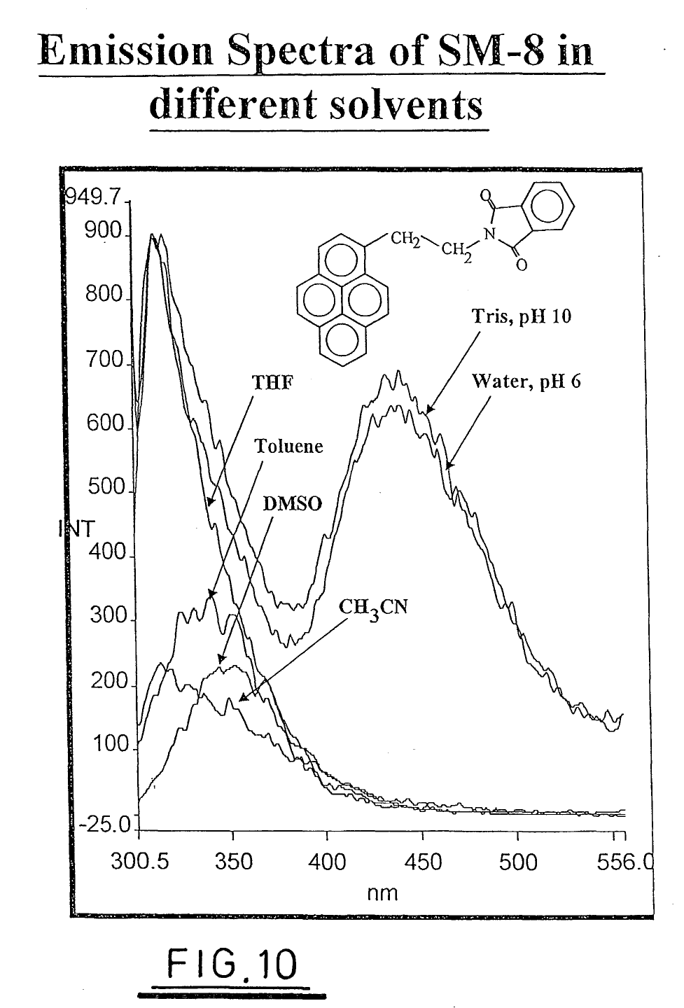

FIG. 9 shows emission spectra of SM-3 (10⁻⁵ M) in 0.01 M Tris buffer, pH 9.0, pH 7.0 and pH 6.5, as indicated (excitation wavelength was 342 nm). The λₘₐₓ for monomer emission was 377 nm for SM-3. An additional intense emission band with λₘₐₓ of 482 nm was observed for SM-3, which is attributed to intramolecular exciplex formation between Pyr and DMA moieties of SM-3. It is seen from FIG. 9 and Table 5 that at pH 6.5 the normalised integral intensity of the exciplex emission for SM-3 was 0.89.

TABLE 5
Fluorescence characteristics of monomer and exciplex emission obtained
for SM-3 in water.

| | Dielectric | λₘₐₓ (nm) | λₘₐₓ (nm) | | |
|---|---|---|---|---|---|
| Solvent | constant (debye) | monomer | exciplex | I_{M}^{c} | I_{EX}^{d} |
| Tris pH 9.0 | 78.54^{a} | 377 | 482 | 0.05 | 0.95 |
| pH 6.5 (tris) | 78.54^{a} | 377 | 482 | 0.11 | 0.89 |

^{a}Dielectric constant was taken from ²⁶;
^{c}Normalised integral intensity of the monomer emission, relative to exciplex
^{d}Normalised integral intensity of the exciplex emission, relative to monomer

### 3. EXAMPLE 3

### Study of SM-8

3.1 Exciplex Formation by SM-8

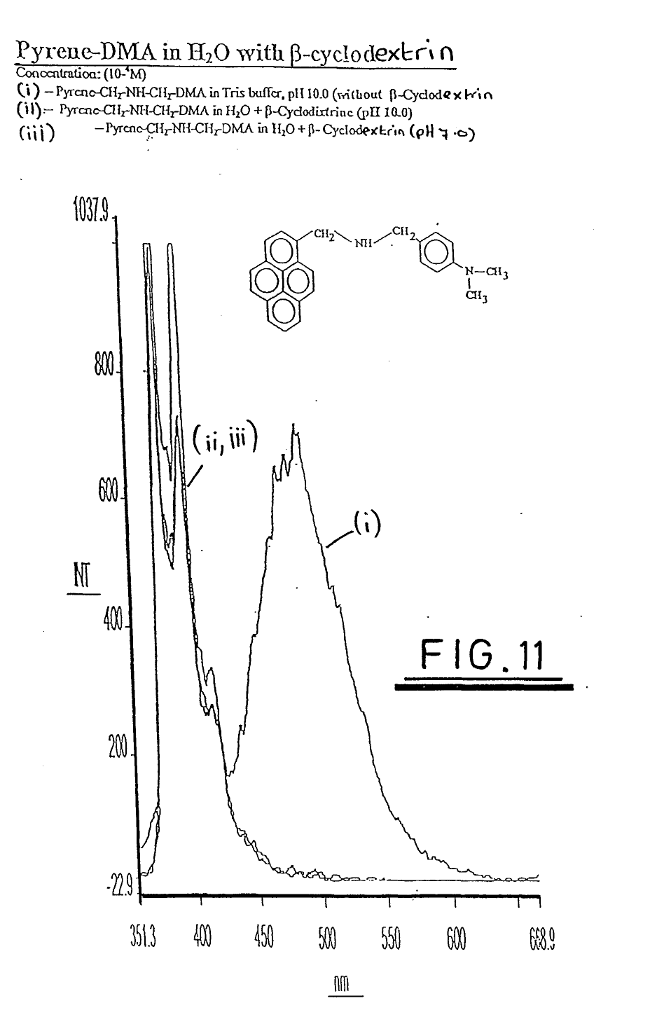

SM-8 also gives an intramolecular exciplex which emits in aqueous media (see FIG. 10).

### 4. EXAMPLE 4

### Influence of Linker Structure on Ability to form Exciplexes

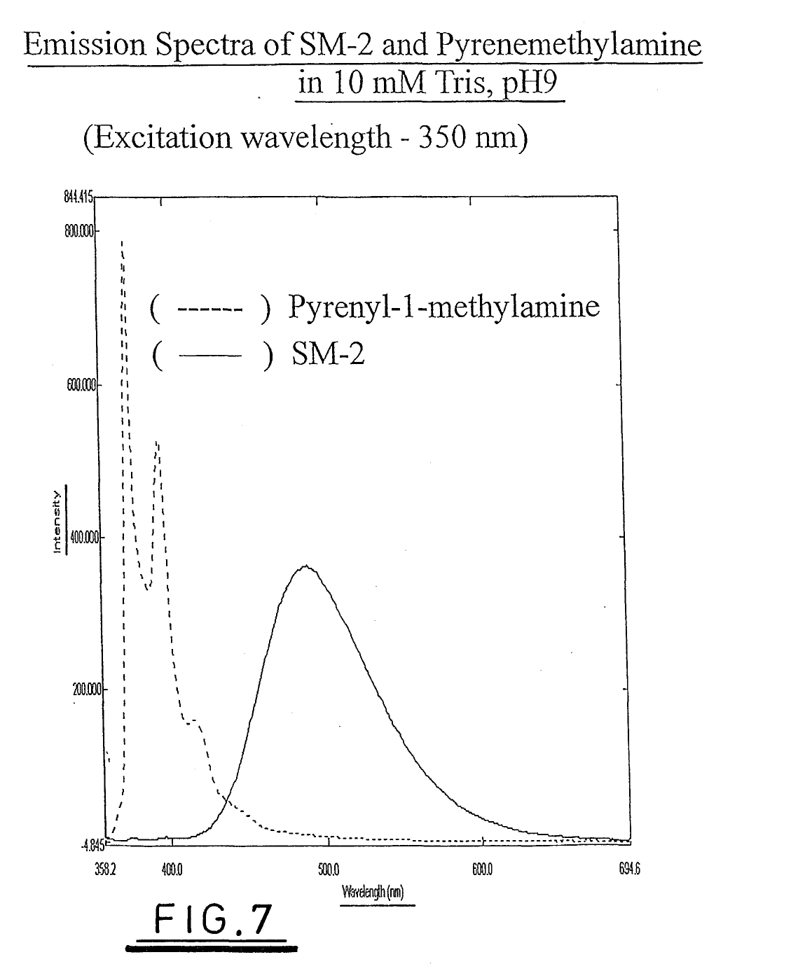

The influence of linker flexibility on exciplex formation was first studied for SM-1 and SM-2 in toluene (FIG. 6) at concentrations of 10⁻⁵ M. Comparison of the emission spectra of SM-1 (FIG. 6) (dashed curve) and SM-2 (FIG. 6) (solid curve) showed that the replacement of the flexible —CH₂—NH—CH₂— linker by the more rigid —CH₂—NH—CO— linker (with planar trans amide group) resulted in complete loss of exciplex emission for Pyr-CH₂—NH—CO-DMA. These experimental observations are in accord with the data obtained by molecular modelling for SM-1 and SM-2 (see Example 5). Similar results were obtained for SM4 in toluene and THF (FIGS. 4 and 5, respectively). SM3 and SM4 are analogues, but SM-3 possesses a flexible —CH₂—NH—CH₂— linker instead of a rigid —CH₂—NH—CO— linker as in SM4.

It should be emphasised that both SM-1 and SM-4 displayed different and completely surprising behaviour in aqueous conditions compared to non-polar solvents.

Other SM systems (SM-5, SM-6 and SM-7) with —CH₂—NH—CO— linker groups were tested for their abilities to form exciplexes in toluene. None of these compounds showed exciplex formation, even in non-polar conditions. The reason for this is the presence of the non-flexible —CH₂—NH—CO— linker group connecting the Pyr with DMA or DMN moieties. An additional reason for the SM-5 and SM-6 compounds may be the substitution of —N—(CH₃)₂ groups by the —NH₂ and —NH—(CH₃) groups in former and latter, respectively. It was shown in a separate experiment that the ability of aniline derivatives to form an intermolecular exciplex with pyrene decreased in the order: DMA, aniline, DEA.

### 5. EXAMPLE 5

### Molecular Modelling and Compound Design

Compound design was performed with the assistance of molecular modelling using SYBYL 6.4.2 software (TRIPOS force field) on a Silicon Graphics Indy R4400 workstation. The structural parameters and charge distribution for the components of the system were calculated using the MOPAC module (SYBYL 6.4.2). All molecular modelling was effected without explicit inclusion of solvent.

It was appeared by computer modelling that linker length and chemical structure could be crucial for exciplex formation. Thus, a short linker group —CH₂—NH—CO— containing a planar, non-flexible amide bond prevents the formation of any stacking orientation between the two partners for the Pyr-CH₂—NH—CO-DMA system (SM-1).

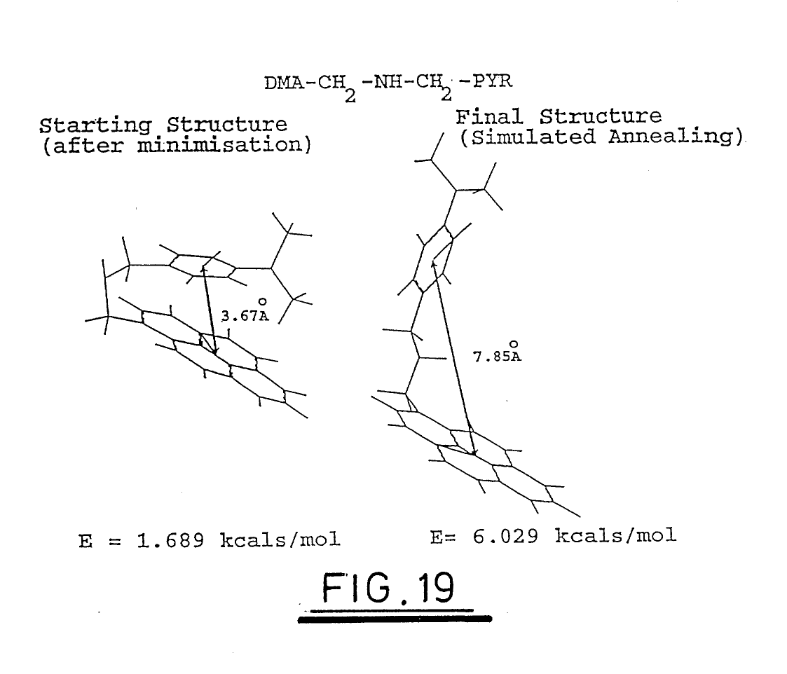

FIG. 18 (left) shows the starting structure of the SM-1 molecule with a restrained parallel orientation of the Pyr and DMA partners which are 3.04 Å apart. It should be noted that this artificial, restrained structure possesses a very high total energy (E=25.802 kcal/mol) due to high values of Angle Bending energy, and especially of Torsional Energy terms. Minimisation of this starting structure (initial Simplex optimisation, followed by the Powell method with a gradient of 0.001 kcalmol⁻¹, TRIPOS force field) resulted in a low-energy final structure (FIG. 18 (right)) with non-stacking orientation of Pyr and DMA partners that are 7.7 Å apart (E=7.29 kcals/mol). This result is consistent with the view that parallel orientation of the donor and acceptor partners favours exciplex emission. This is geometrically forbidden for Pyr-CH₂—NH—CO-DMA, in turn suggesting that exciplex formation is likely to be less favoured in this case.

The substitution of the —CO group by the more flexible —CH₂ group increases the ability of Pyr-CH₂—NH—CH₂-DMA (SM-2) to form a stacking structure with parallel orientation of the closely located Pyr and DMA components, perhaps due to a higher degree of conformational freedom of linker group.

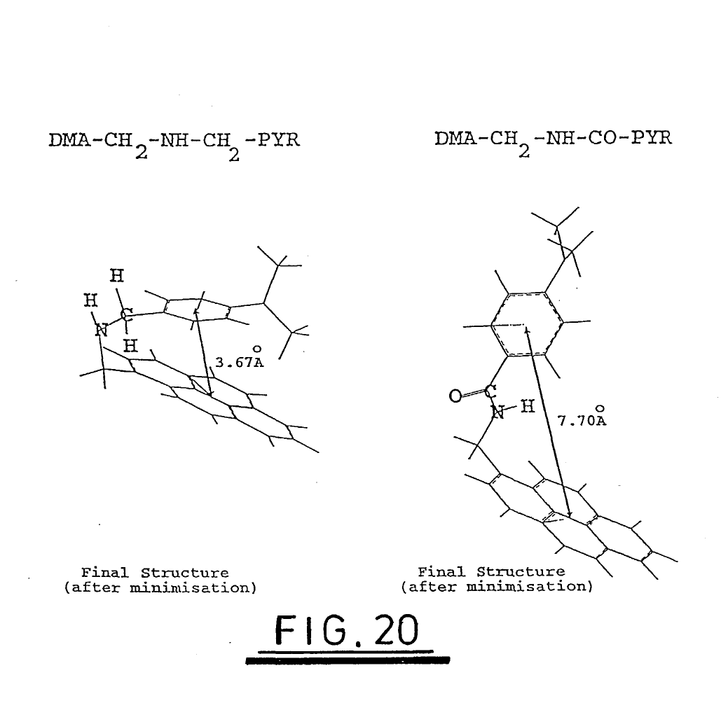

FIG. 19 shows the two low-energy structures of SM-2 representing the stacking and non-stacking orientation of the Pyr and DMA partners, respectively. It is seen that a stacking structure with close location of exciplex partners (3.67 Å) was characterised with a lower total energy than the non-stacking structure (the distance between Pyr and DMA was 7.85 Å). This result clearly suggests the potential ability of Pyr-CH₂—NH—CH₂-DMA to form an exciplex structure.

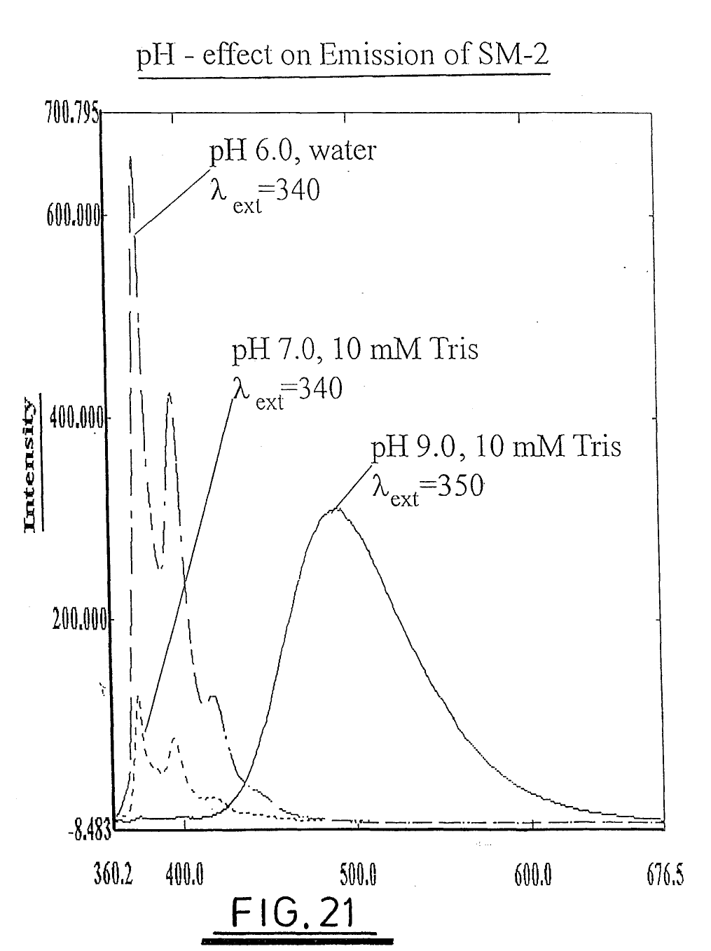

FIG. 20 summarises the results of molecular modelling obtained for SM-2 (left) and SM-1 (right), showing the most favourable low-energy conformations for these molecules.

Thus, the results of molecular modelling are consistent with the supposition that the linker groups need to be flexible, and possess a high degree of rotational freedom to allow parallel orientation of the partner chromophores. Rigid, non-flexible fragments in linker groups impose strict conformational rules that in general disfavour formation of a putative parallel exciplex structure.

### 6. EXAMPLE 6

### Absorption and Excitation Spectra of SM-1, SM-2 and SM-3

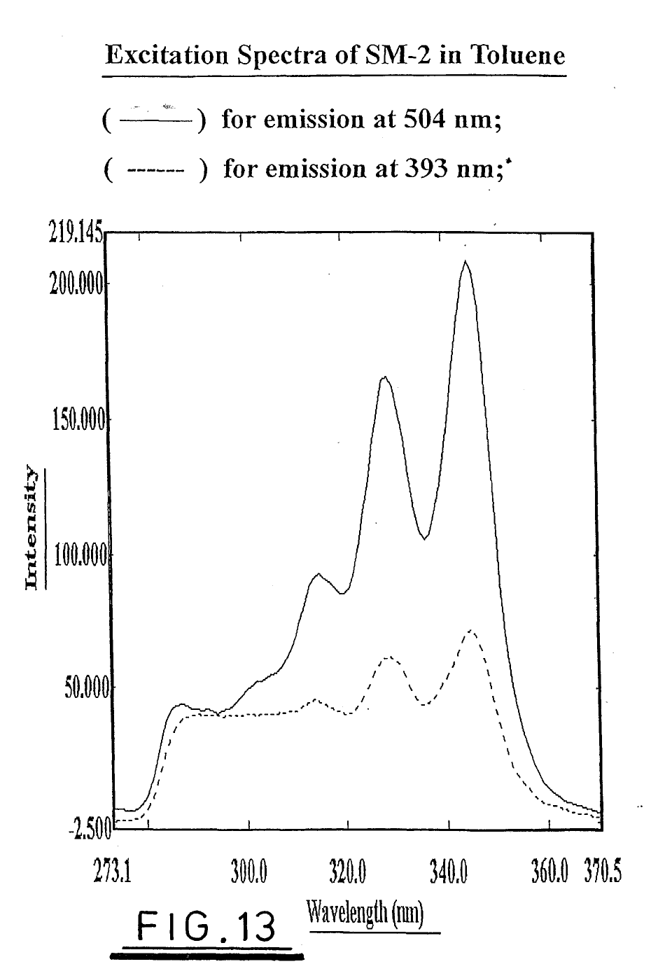

To characterise the excited and ground states of some SMs we recorded the UV-visible absorption spectra as well as excitation spectra for both monomer and exciplex band emission (FIGS. 12-17).

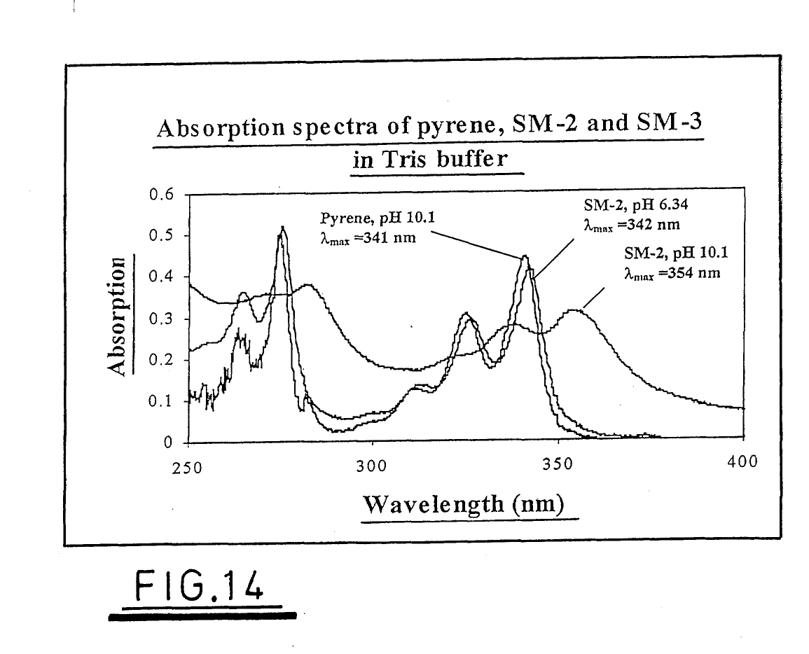

FIG. 12 shows absorption spectra of SM-2, SM-1, 1-methylamino-pyrene and naphthalene recorded in toluene at 10⁻⁵ M concentration. It is seen that low-wavelength bands of the pyrene moiety (310-350 nm) of both SM-1 and SM-2 show close spectral similarity to the respective absorption band of 1-methylamino-pyrene, apart from the slight red-shift of the λₘₐₓ of both SM-1 and SM-2 (346 nm) as compared with λₘₐₓ of free 1-methylamino-pyrene (344 nm), which may be attributed to the difference in chemical structures of these compounds. This result indicates the similarity of ground states for the pyrene moieties for SM-1, SM-2 and 1-methylamino-pyrene. These data are in accordance with the excitation spectrum of SM-2 recorded in toluene (FIG. 13). The excitation spectrum for the exciplex fluorescence emission band at 504 nm is identical to the excitation spectrum for monomer fluorescence emission band at 393 nm, showing that the exciplex responsible for the lower-wavelength fluorescence band is dissociated in the ground state.

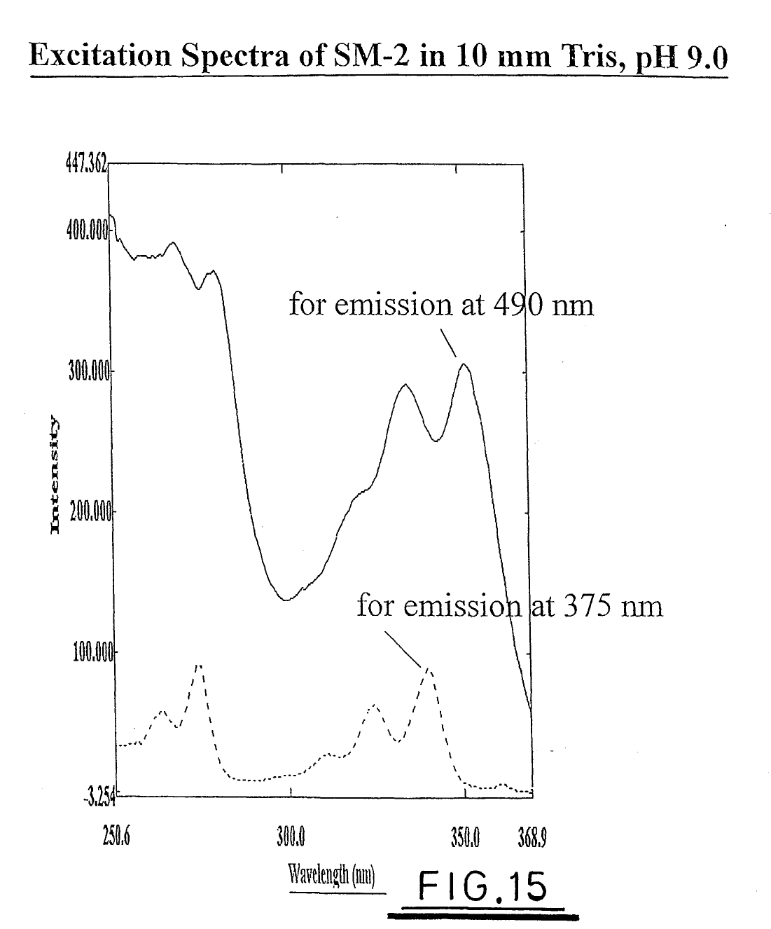

A slightly different situation and thus surprising was found for SM-2 in aqueous solutions. The UV-visible spectrum of SM-2 (FIG. 14) in 10 mM Tris buffer (pH 9.0) showed significant spectral broadening and a 13 nm red-shift of the lower-wavelength band (λₘₐₓ, 354 nm) compared with 1-methylamino-pyrene (λₘₐₓ, 341 nm, black curve). However, no additional absorption band was observed for SM-2 under the above conditions. It should be noticed that no spectral broadening and no significant red-shift were observed for the absorption spectrum of SM-2 recorded at pH 6.34 (magenta) when no exciplex was formed. These data suggest some charge-transfer interactions between donor and acceptor groups in their ground states. Compare reports for intramolecular exciplexes formed from anthracene²⁸.

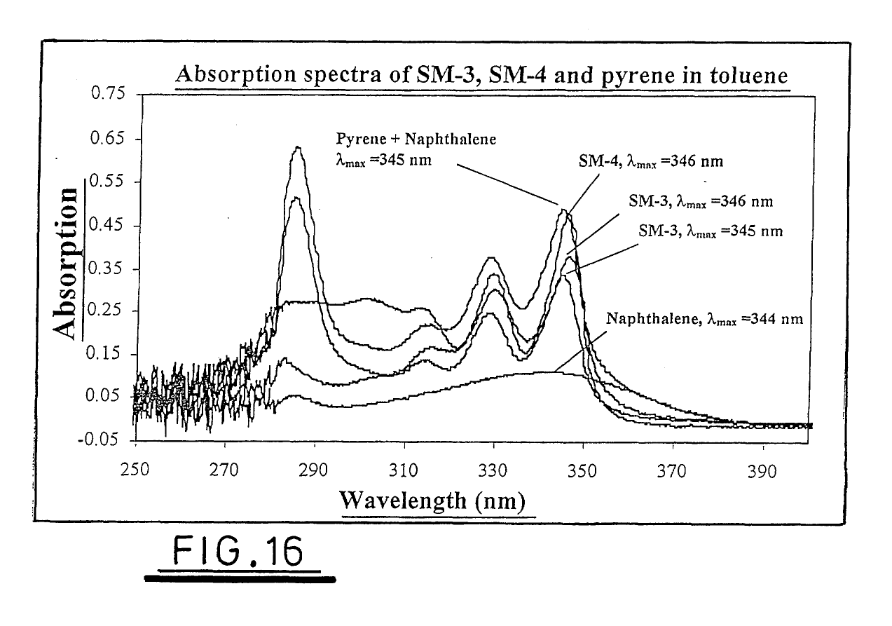

These data are correlated with the excitation spectra recorded for SM-2 in 10 mM Tris buffer (pH 9.0) (FIG. 15). The excitation spectrum for the exciplex fluorescence emission band at 490 nm is significantly broadened and red-shifted compared to the excitation-spectrum for the monomer fluorescence emission band at 375 nm, showing the charge-transfer nature of the exciplex in aqueous solutions.

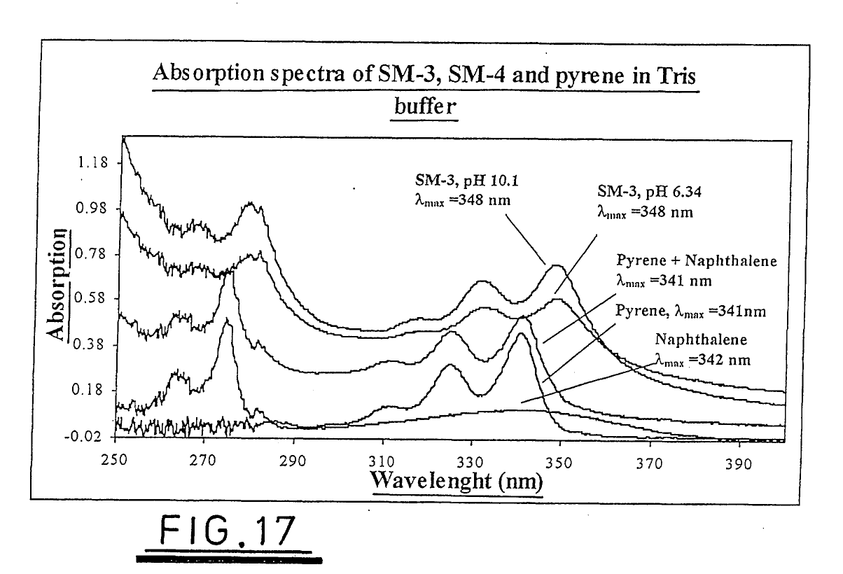

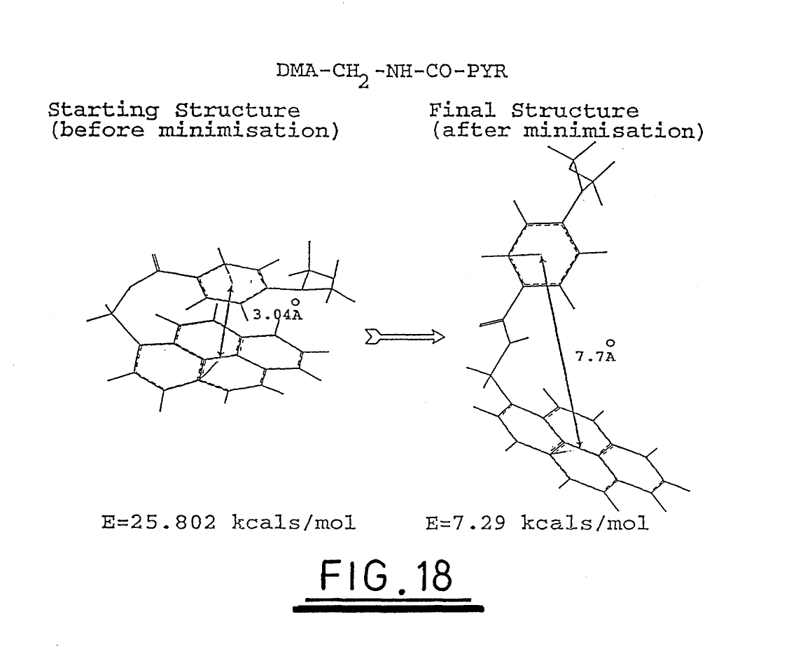

Similar results were obtained for SM-3 both in toluene and aqueous solution (FIGS. 16 and 17, respectively).

### 7. EXAMPLE 7

### Labelling of Primers

(i) The 8-mer oligodeoxyribonucleotide pTGTTTGGC was condensed through its 5′-phosphate site with N-(2-aminoethyl)-N-(4-dimethylaminobenzyl)-N-(1-pyrenyl)amine (3) to produce the labelled oligonucleotide designated herein as 3-pTGTTTGGC. The synthetic procedures used were as described in Appendix A.

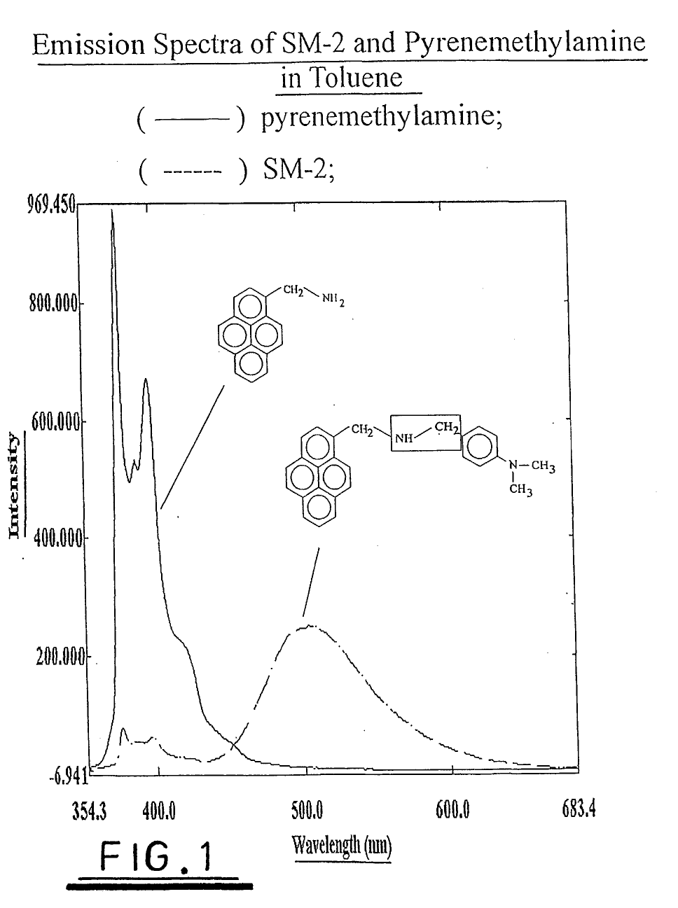

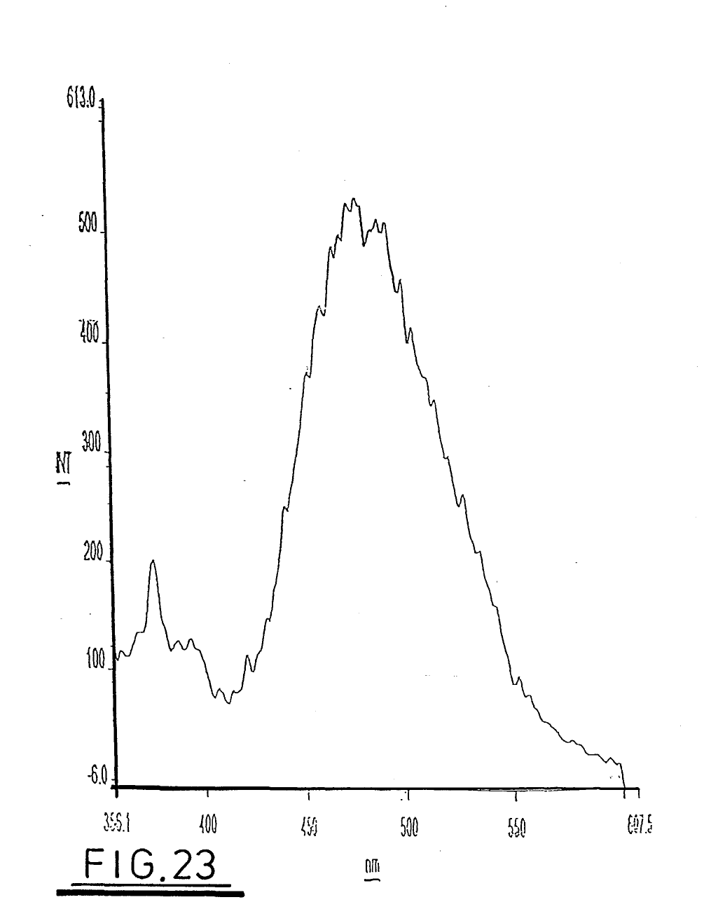

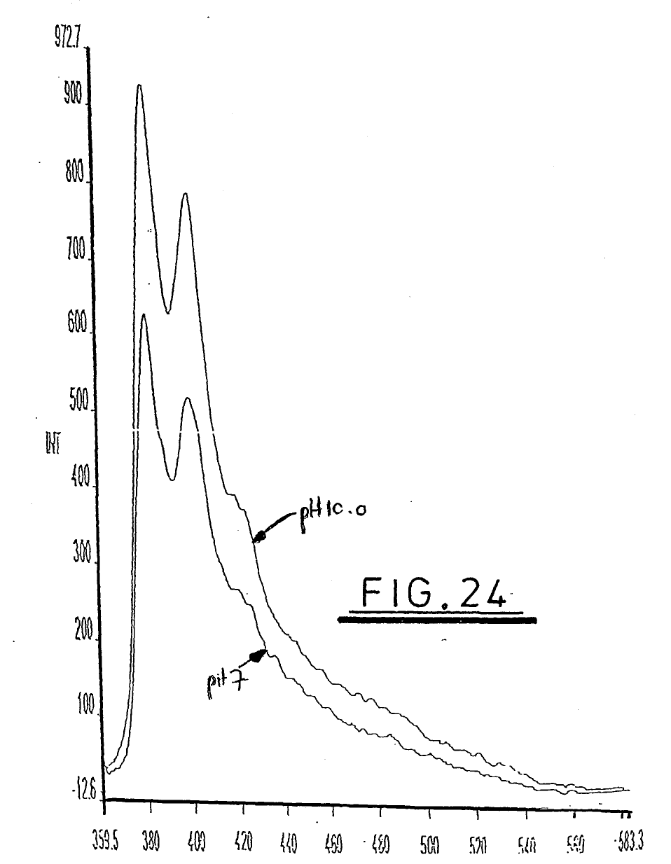

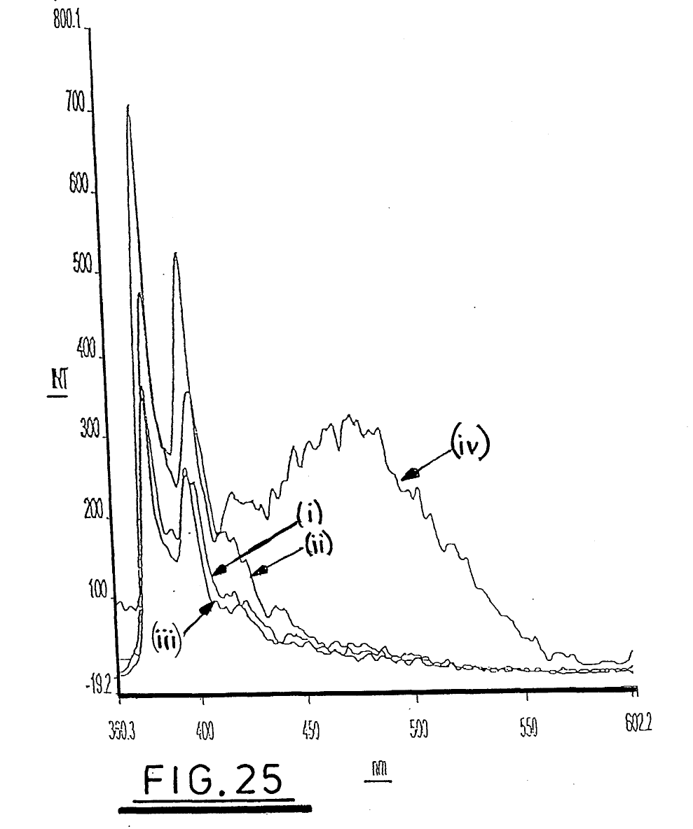

The exciplex emission spectrum of 3 in pH 9 tris buffer is illustrated in FIG. 23. For 3-pTGTTTGGC a long tail of emission was seen in the region of 480 nm, with only a weak peak in this region at pH 7 or 10 (FIG. 24). The effects of added organic solvent on the exciplex emission of 3-pTGTTTGGC (FIG. 25) were tested: the addition of (i) acetonitrile or (ii) methanol (to 50% v/v) to 3-pTGTTTGGC in pH 8.5 Tris buffer gave no significant increase in emission at 480 nm, although there was some change detected for (iii) tetrahydrofuran. The spectrum of 3-pTGTTTGGC in (iv) pure tetrahydrofuran (Figure 0.25) shows strong emission at 485 nm, typical of the exciplex.

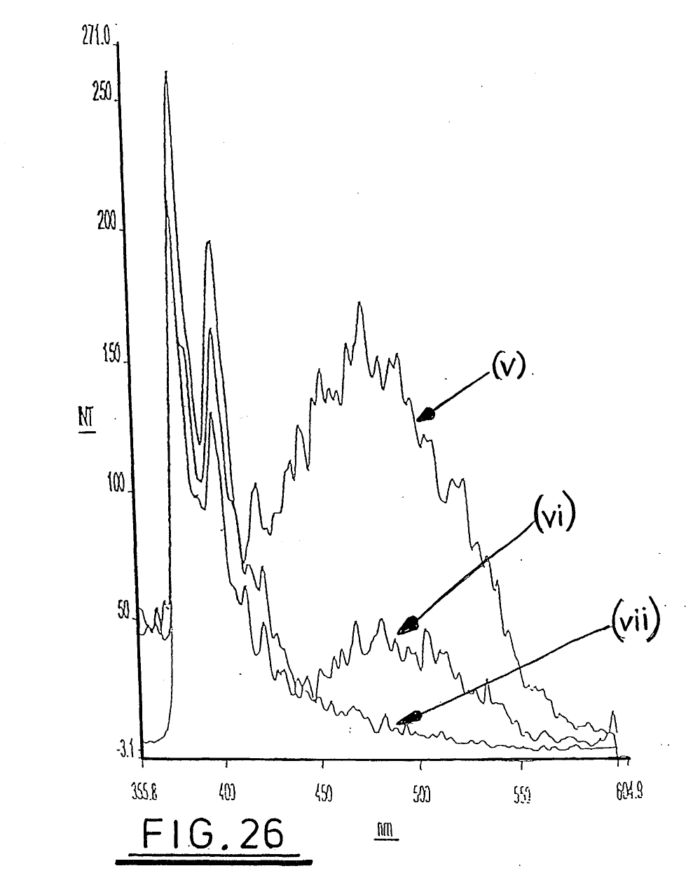

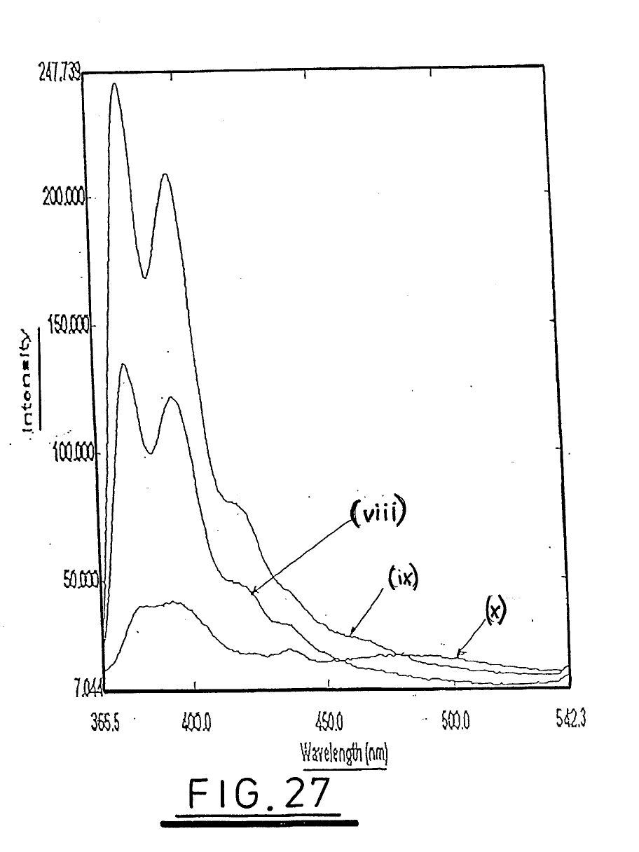

(ii) The 8-mer dCGATTCTGp was labelled on the 3′ phosphate with compound 3 to produce the labelled oligonucleotide designated herein as dCGATTCTGp-3. The emission spectra of dCGATTCTGp-3 are shown in FIG. 26: the exciplex emission band around 485 nm is clearly seen in (v) pure tetrahydrofuran and in (vi) a medium prepared from 0.01M Tris buffer at pH 8.8 (10% v/v) and tetrahydrofuran (90% v/v). The exciplex emission spectrum in 0.01 m Tris buffer at pH 8.8 is shown in FIG. 4 as curve (vii). With reference to FIG. 27, the exciplex emission band at 480 nm is seen to increase in distinctness on going from (viii) 50, to (ix) 75 to (x) 92.5% THF in the Tris buffer pH 8.8 (in these experiments the solution was diluted by addition of THF and so the concentration of fluorophore also decreased).

### SUMMARY

1. A number of examples are given whereby intramolecular exciplexes can be constructed such that their exciplex properties are not quenched in aqueous or other highly polar media.

2. This allows the use of aqueous media to make use of any property of the exciplex that previously was restricted to non or low percentage aqueous environments, such as lasers, dyes to act as fluorescent or circular dichroism detectors when the intramolecular exciplex analogues have been used to label other materials including, proteins, nucleic acids and (oligo)nucleotides and their analogues, such as PNAs, glycoproteins, solid materials including polymers, ceramics, transistors, semiconductors, insulators, conductors, glasses.

3. It appears from the observed data that the relative fluorescence emission in the region of the inflexion in the curve is MORE sensitive to pH than predicted by the Henderson-Hasselbach equation. This is even more surprising and novel and means that the system may provide the basis of an even more pH-sensitive pH device or pH switch. The solid curve shown in the Figure indicates what would be the behaviour on pH change if Henderson-Hasselbach behaviour were followed.

4. In contrast to SM-2, the ability of SM-3 to give exciplex emission is less sensitive to the pH of the solution. It is seen from FIG. 7 and Table 1 that even at pH 6.5 the normalised integral intensity of the exciplex emission for SM-2 was 0.89.

5. Not all exciplexes which we have made and shown to emit in aqueous media show pH effects and thus the observations above are surprising (compare SM-8, FIG. 10).

4. Appendix A. Synthetic Methods

A.1 Synthesis of SM-1 and SM-2

(i) 4-Dimethylamino-N-(1-pyrenemethyl)benzamide, SM-1

A solution of 1-pyrenemethylamine hydrochloride (0.5 g, 1.865 mmol) in dichloromethane (150 ml) was treated with triethylamine (0.45 g, 4.47 mmol) and stirred for 30 min at room temperature. 4-Dimethylaminobenzoyl chloride (0.34 g, 1.865 mmol) in dichloromethane (50 ml) was added dropwise over 30 min. and the mixture stirred for 5 hrs until TLC (DCM/EtOAc, 9:1) indicated completion. The reaction mixture was washed successively with 1N HCl (1×100 ml), 1N NaHCO₃ (3×100 ml), H₂O (100 ml) and brine (100 ml). The organic layer was dried over anhydrous Na₂SO₄. Evaporation of solvent afforded crude product which was recrystallised from ether to give a white solid (0.358 g, 50.59%); m.p: 204-206° C. ¹H-NMR (δH, CDCl₃): 2.97 (s, 6H, —NCH₃); 5.34 (d, 2H, PyCH₂N—); 6.34 (bs, 1H, —NHCO—); 6.69 (d, 2H, —Ar); 7.71 (d, 2H, —Ar); 7.97-8.36 (m, 9H, —Py), ¹³C-NMR (δ_{C}, CDCl₃): 40.31; 42.63; 111.83; 123.44; 124.69; 124.88; 125.31; 126.25; 127.31; 127.56; 128.56; 129.25; 129.50; 130.81; 131.88; 167.19.

(ii) N-(4-Dimethylaminobenzyl)-N-(1-pyrenemethyl)amine, SM-2

To a solution of 4-dimethylamino-N-(l-pyrenemethyl)benzamide (0.1 gr, 0.26 mmol) in dry ether (15 ml) was added LiAH₄ (0.075 g, 2 mmol). The mixture was refluxed until TLC monitoring showed consumption of all of the starting material. The mixture was then stirred at room temperature overnight. After washing with water (2×20 ml), the ethereal solution was dried over anhydrous MgSO₄. The resulting oily residue after evaporation was purified by flash chromatography on a column of silica gel with ether to afford a light yellow product as an oil (0.07 g, 70%). The oil was dissolved in DCM/ether and HCl(g) was bubbled through to form the corresponding dihydrochloride salt as a white solid; R_{f}: 0.21 Et₂O). ¹H-NMP (δ, CDCl₃) 2.09 (bs, 1H, —NH); 2.89 (s, 6H, 2-NCH₃); 3.83 (s, 2H, —NCH₂Py); 4.41 (s, 2H, —NCH₂Ar); 6.67-6.74 (dd, 2H, —Ar); 7.21-7.27 (dd, 2H, —Ar); 7.80-8.47; (m, 9H, -Py).

A.2 Synthesis of SM-5

4-Amino-2,3,5,6-tetrafluoro-N-(1-pyrenemethyl)benzamide, SM-5

A solution of 1-pyrenemethylamine hydrochloride (0.2 g, 0.72 mmol) in DCM (15 ml) and triethylamine (0.11 ml, 0.86 mmol) was stirred for 30 min at room temperature. To this mixture was added 4-amino-2,3,5,6-tetrafluorobenzoic acid (0.157 g, 0.76 mmol,) in DCM (15 ml) and 4-dimethylaminopyridine (0.09 g, 0.74 mmol). Finally N,N-diisopropylcarbodiimide (0.12 ml, 0.77 mmol) was added drop-wise at 0° C. The mixture was refluxed for 3 hrs and stirring continued overnight at room temperature. After washing with NaHCO₃ (10%, 30 ml) and water (2×30 ml), the organic layer was dried over MgSO₄. Evaporation of solvent and purification of residue afforded the pure product as a cream solid (0.19 g, 60%), R_{f}: 0.55 (DCM/EtOAc, 17:1). ¹H-NMR (δ, CDCl₃): 4.19 (bs, 2H, —NH₂); 5.35-5.36 (d, 2H, PyCH₂N); 6.28 (bs, 1H, —NHCO—); 8.01-8.33 (m, 9H, -Py).

A.3 Synthesis of SM-7

4-Amino-3-nitro-N-(1-pyrenemethyl)benzamide, SM-7

A solution of 1-pyrenemethylamine hydrochloride (0.1 g, 0.36 mmol) in dichloromethame (10 ml) and triethylamine (0.05 ml, 0.36 mmol) was stirred for 30 min at room temperature. To this mixture was added 4-amino-3-nitrobenzoic acid (0.07 g, 0.38 mmol) in dichloromethane (10 ml) and 4-dimethylaminopyridine (0.044 g, 0.36 mmol). Finally N,N-diisopropylcarbodiimide (0.06 ml, 0.38 mmol) was added dropwise at 0° C. The mixture was refluxed for 3 hrs and stirring was continued at room temperature overnight. After washing with NaHCO₃ (10%, 20 ml) and water (2×20 ml), the organic layer was dried on MgSO₄. Evaporation of solvent and purification of the residue with dichloromethane/EtoAc (3:1) on a column of silica gel afforded the pure product as a yellow solid (0.09 g, 61%); R_{f} 0.44 (DCM/EtOAc, 3:1). ¹H-NMR (δ, Acetone-d₆): 4.95-5.02 (m, 2H, —NH₂); 5.21-5.22 (d, 2H, —CH₂N); 6.94-6.98 (d, 1H, —Ar); 7.22 (bs, 1H, —NHCO); 7.86-8.42 (m, 10H, -Py, —Ar); 8.56-8.59 (d, 1H, —Ar).

A.4 Synthesis of SM-3 and SM-4.

(i) 4-N-Dimethylamino-(1-pyrenemethyl)naphthamide, SM-4

A solution of 1-pyrenemethylamine hydrochloride (0.2 g, 0.72 mmol) in DCM (20 ml) was treated with triethylamine (0.11 ml, 0.79 mmol) and stirred for 30 min at room temperature. 4-Dimethylamino-naphthoic acid (0.194 g, 0.76 mmol, in DCM (20 ml) and 4-dimethylaminopyridine (0.088 g, 0.72 mmol) were added. To this solution, N,N-diisopropylcarbodiimide (0.12 ml, 0.77 mmol) was added drop-wise at 0° C. under nitrogen and the reaction mixture refluxed for 3 hrs. The mixture was stirred overnight at room temperature under drying tube and then washed with 1N NaHCO₃ (50 ml) and H₂O (2×50 ml). The organic layer was dried over anhydrous MgSO₄. Evaporation of solvent gave the crude product and column chromatography on silica gel with DCM/EtOAc (10:1) afforded the pure product.

(ii) N′-4-Dimethylaminonaphthyl-N-(1-pyrenemethyl)amine, SM-3

In a 50 ml flask, LiAH₄ (0.075 g) was added to a solution of 4-dimethylamino-N-(1-pyrenemethyl)naphthamide (SM-3, 0.17 g, 0.4 mmol) in dry ether (30 ml) and dry THF (15 ml). The mixture was refluxed for 7 hrs. TLC monitoring showed all of the starting material was consumed. The reaction mixture was left stirred overnight at room temperature. Aqueous ammonium chloride was added and the extracted ethereal layer dried on MgSO₄. Column chromatoghraphy of the residue from evaporation on silica gel (DCM eluent) gave the pure product as a light brown liquid (0.065 g, 40.6%), R_{f} 0.14 (DCM/EtOAc, 20:1). ¹H-NMR (δ_{H}, CDCl₃): 1.32 (bs, 2H, —NH₂); 3.11 (t, 2H, ArCH₂); 3.20 (t, 2H, —CH₂NH); 7.49-7.66 (m, 5H, —Ar); 7.80 (d, 1H, —Ar); 8.06 (d, 1H, —Ar); 8.72-8.59 (dd, 2H, —Ar).

A.5 Synthesis of SM-6

4-Dimethylamino-3,5-dinitro-N-(1-pyrenemethyl)benzamide, SM-6

A solution of 1-pyrenemethylamine hydrochloride (0.2 g, 0.72 mmol) in DCM (20 ml) was treated with triethylamine (0.11 ml, 0.79 mmol) and stirred for 30 min at room temperature. 4-Dimethylamino-3,5-dinitrobenzoic acid (0.194 g, 0.76 mmol) in DCM (20 ml) and 4-dimethylaminopyridine (0.088 g, 0.72 mmol) were added. To this solution, N,N-diisopropylcarbodiimide (0.12 ml, 0.77 mmol) was added drop-wise at 0° C. under nitrogen and the reaction mixture refluxed for 3 hrs. The mixture was stirred overnight at room temperature under drying tube and then washed with 1N NaHCO₃ (50 ml) and H₂O (2×50 ml). The organic layer was dried over anhydrous MgSO₄. Evaporation of solvent and column chromatography of the crude product with hexane/DCM (1:4) afforded the pure product as a yellow solid (0.09 g, 24.3%); m.p.: 221-224° C. R_{f}: 0.23 (CDM). ¹H-NMR (δ_{H}, CDCl₃): 2.82 (s, 6H, 2-NCH₃); 5.32 (d, —CH₂NH); 7.96-8.28 (m, 11H, —Ar, -Py).

Synthesis of 9-((N,N-dimethylamino)ethyl)phenanthrene (SM-9)

To prepare 9-((N,N-dimethylamino)ethyl)phenanthrene (SM-9), 2-(9-phenanthrenyl)ethanol was synthesised from 9-bromophenanthrene via the 9-phenanthrenyl magnesium bromide^{29;30} and ethylene oxide^{30;31}. This was converted to 2-(9-phenanthrenyl)ethyl chloride using thionyl chloride³¹, Scheme X. Reaction of 2-(9-phenanthrenyl)ethyl chloride with potassium phthalimide in the presence of hexadecyltributylphosphonium bromide afforded the corresponding phthalimide, which was hydrolysed to 2-(9-phenanthrenyl)ethylamine using hydrazine hydrate³², Scheme XI. Dimethylation of 2-(9-phenanthrenyl)ethylamine gave SM-9.

(i) 2-(9-Phenanthryl)ethanol

9-Bromophenanthrene (2 g, 7.8 mmol), magnesium turning (0.2 g) and a catalytic amount of I₂ were suspended in dry THF (20 ml). The mixture was refluxed until all the magnesium had dissolved (1 hr), the mixture cooled to −10° C. and 1.4 ml of ethylene oxide (0.48 g, 10.8 mmol) in dry ether (1.4 ml from a solution contains 34 gr ethylene oxide in 100 ml dry ether) was added. After stirring at 0° C. for 30 min and then at 40° C. for 30 min, the solution was refluxed for 1 hr, cooled to room temperature and then concentrated. Addition of 1N HCl (10 ml) was followed by extraction with dichloromethane (2×20 ml). The organic layer was washed with 1N NaOH (20 ml) and H₂O and then dried over anhydrous MgSO₄. Evaporation of solvent and chromatography of crude product on silica gel with hexane/dichloromethane (3:7) afforded the pure product as a white solid (1.2 g, 69.4%); m.p.: 86-87° C. R_{f}: 0.34 (DCM). ¹H-NMR (δ, CDCl₃):1.53 (bs, 1H, OH); 3.42 (t, 2H, —CH₂—CH₂—O); 4.07 (t, 2H, —CH₂—CH₂—O); 7.58-7.72 (m, 5H, —Ar); 7.84-7.90 (d, 1H, —Ar); 8.11-8.16 (d, 1H, —Ar); 8.66-8.72 (d, 1H, —Ar); 8.74-8.80 (d, 1H, —Ar).

(ii) 2-(9-Phenanthryl)ethyl Chloride

To a cold mixture of 2-(9-phenanthryl)ethanol (0.6 g, 2.7 mmol) and N,N-dimethylalanine (0.35 ml) in dry toluene (2 ml) cooled on ice water thionyl chloride (0.23 ml, 3.15 mmol) was added drop-wise. The red solution was refluxed until the evolution of sulfur dioxide ceased. On addition of water (20 ml) an organic layer separated and was isolated with ether(30 ml). After evaporation of ether and column chromatography on silica gel with hexane, the corresponding pure product was obtained as a white solid (0.5 g, 77.0%); m.p.: 82-84° C. ¹H-NMR (δ, CDCl₃): 3.54-3.62 (t, 2H, —CH₂CH₂Cl); 3.86-3.93 (t, 2H, —CH₂CH₂Cl); 7.57-7.69 (m, 5H, -Phe); 7.83-7.85 (m, 1H, -Phe); 8.04-8.06 (m, 1H, -Phe); 8.65-8.67 (d, 1H, -Phe).

(iii) 2-(9-Phenanthrenyl)ethylphthalimide

A toluene solution (2 ml) of 2-(9-phenanthrenyl)ethyl chloride (0.241 g, 1 mmol) and hexadecyltributylphosphonium bromide (0.051 g, 0.1 mmol), and potassium phthalimide (0.231 g, 1.25 mmol) were placed in a 5 ml round-bottomed flask equipped with reflux condenser and magnetic stirrer, and heated at 100° C. (bath temperature) under argon atmosphere with stirring. The extent of reaction was monitored by following the disappearance of starting material. After 20 hrs (95% conversion) water (30 ml) was added to the cooled reaction mixture which was then extracted with ether (3×30 ml). The combined ethereal layer was dried over MgSO₄ and evaporated under vacuum. The crude product was purified by column chromatography on silica gel with hexane/DCM (50:50) as eluent. The pure product was obtained as a white solid (0.24 g, 56.6%), m.p: 140-143° C., R_{f}: 0.31 (hexane/DCM).

(iv) 4-N′,N′-dimethylamino-N-(1-pyrenemethyl)naphthamide

Using the method described for 4-dimethylamino-3,5-dinitrobenzoic acid, the pure product (0.283 g, 80%) was obtained as a white solid after column chromatography on silica gel with DCM/EtOAc (10:1) as eluent. 4-N′,N′-dimethylaminonaphthyl-N-(1-pyrenemethyl)amine, SM-3

In a 50 ml flask, LiAH₄ (0.075 g) was added to a solution of 4-dimethylarnino-N-(1-pyrenemethyl)naphthamide (0.17 g, 0.4 mmol) in dry ether (30 ml) and dry THF (15 ml). The mixture was refluxed for 7 hrs. TLC monitoring showed all of the starting material was consumed. The reaction mixture was left overnight in room temperature while it was stirred. An aqueous solution of ammonium chloride was added to the mixture and the ethereal layer extracted and dried over MgSO₄. Column chromatography of the residue on silica gel with DCM as eluent gave the pure product as a light brown liquid (0.065 g, 40.6%), R_{f}:0.14 (DCM/EtOAc, 20:1), ¹H-NMR (δ_{H}, CDCl₃): 1.32 (bs, 2H, —NH₂); 3.11 (t, 2H, ArCH₂); 3.20 (t, 2H, —CH₂NH); 7.49-7.66 (m, 5H, —Ar); 7.80 (d, 1H, —Ar); 8.06 (d, 1H, —Ar); 8.72-8.59 (dd, 2H, —Ar).

Synthesis of N-(2-aminoethyl)-N-(4-dimethylaminobenzyl)-N-(1-pyrenyl)amine

This was carried out according to the following Scheme

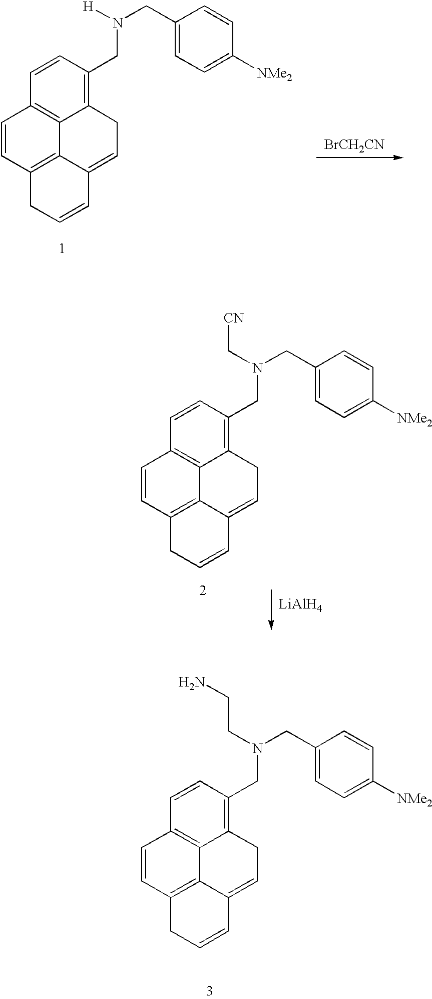

Synthesis of N-(cyanomethyl)-N-(4-dimethylaminobenzyl)-N-(1-pyrenyl)amine, 2

Bromoacetonitrile (0.62 mg, 0.52 mmol 0.035 ml) was added drop-wise to a stirred solution of N-(4-dimethylaminobenzyl)-N-1-pyrenyl)amine (0.19 g, 0.52 mmol) and potassium carbonate (0.071 g, 0.52 mmol) in acetone (0.5 ml) at 0° C. The reaction mixture was stirred at room temperature for 3 h and then the solvent removed in vacuo. The resultant solid was triturated with water (15 ml) and extracted with diethyl ether (2×20 ml). The combined organic extracts were washed with water (2×10 ml), dried over anhydrous Na₂SO₄, filtered and evaporated to dryness to give N-(cyanomethyl)-N-(4-dimethylaminobenzyl)-N-(1-pyrenyl)amine (2) as a yellow solid, which after chromatography on silica gel with dichloromethane and hexane as solvent (9:1) was isolated as a white product (0.08 g, 38%).

Synthesis of N-(2-aminoethyl)-N-(4-dimethylaminobenzyl)-N-(1-pyrenyl)amine, 3

To a solution of N-(cyanomethyl)-N-(4-dimethylaminobenzyl)-N-(1-pyrenyl)amine (2, 0.06 g, 0.15 mmol) in dry tetrahydrofuran-(1 ml) was added LiAlH₄ (0.05 g, 1.3 mmol) and the mixture refluxed for 5 h after which the complex was decomposed by adding aqueous potassium hydroxide (2 ml, 40%). This mixture was extracted with diethyl ether (2×15 ml) after addition of a further aliquot of water (1 ml) and the combined ethereal extracts dried over anhydrous Na₂SO₄, filtered and evaporated to dryness to give an oily yellow residue. Chromatography on Varian Mega BondElut SI(2 g silica gel) with dichloromethane and a few drops of aq. NH₄OH yielded 3 as a yellow, oily product (0.05 g, 81%).

DNA Labelling

To give 3-pTGTTTGGC, the 8-mer oligodeoxyribonucleotide pTGTTTGGC was condensed through its 5′-phosphate site with N-(2-aminoethyl)-N-(4-dimethylaminobenzyl)-N-(1-pyrenyl)amine (3) as follows. 3-pTGTTTGGC was synthesised by a two-step procedure. First, the cetyltrimethylammonium salt of 5′pTGTTTGGC was obtained by stepwise addition of 8% aqueous cetyltrimethylammonium bromide (10 μl, 20μl×5) to a solution of the lithium salt of the oligonucleotide (1 μmol) in 0.3 ml of water, with centrifugation on each addition, until no more precipitation was observed. The supernatant was removed, the precipitate dried in vacuo overnight over P₂O₅, and the cetyltrimethylammonium salt of the oligonucleotide (1 μmol) dissolved in 0.4 ml of DMF. Triphenylphosphine (13.2 mg, 50 μmol) and 2′,2′-dipyridyl disulfide (11.2 mg, 50 μmol) were added, and after 10 min 4-N′,N′-dimethylaminopyridine (6.2 mg, 50 μmol) was added. After 15 minutes incubation at 20° C., N-(2-aminoethyl)-N-(4-dimethylaminobenzyl)-N-(1-pyrenyl)amine (3) (5.4 mg, 20 μmol) and triethylamine (281 μl, 20 μmol) was added and the reaction mixture incubated at 25° C. for 1 h, followed by precipitation by 25 ml of acetone containing 2% LiClO₄. The oligonucleotide conjugate (3-pTGTTTGGC) was separated from unreacted precursor (3) by reverse-phase HPLC (0 to 40% acetonitrile gradient) to give total yield of product of 90% based on starting oligonucleotide. The incorporation of the structure from 3 into the oligonucleotide was confirmed by its HPLC behaviour, and by UV-visible specrophotometry, giving a characteristic absorbance at 35Q nm maximally for the chromophore corresponding to 3.

The synthesis of DCGATTCTGp-3 was as described above but using dCGATTCTGp labelled on the 3′ phosphate with compound 3.

### REFERENCE LIST

1. Exciplex and ion pair quenching in a chiral hexahelicene-amine system. J.Photochem.Photobiol.A.Chem 63, 337-347. 1992.

2. Knibbe, H. Charge-transfer complex and solvent-shared ion pair in fluorescence quenching. Letters to the editor 47, 1184-1185. 1967.

3. Gordon, M. and Ware, W. R. (1975) The EXCIPLEX, Academic Press Inc., New York San Francisco London,

4. Birks, J. B. (1973) Organic Molecular Photophysics, John Wiley & Sons; London—New York—Toronto—Sydney,

5. Birks, J. B. (1973) Organic Molecular Photophysics, John Wiley & Sons; London—New York—Toronto—Sydney,

6. Masaki, S., Okada, T., Mataga, N., Sakata, Y., and Misumi, S. On the solvent-induced Changes of electronic structures of intramolecular exciplexes. Bulletin of the Chemical Society of Japan 49(5), 1277-1283. 1976.

7. On the electronic structure of exciplexes in α-(1- or 2-naphthyl)-ω-N,N-dialkylaminoalkanes. Journal of Photochemistry 28, 315-327. 1985.

8. Van Haver, Ph., Helsen, N., Depaemelaere, S., Van der Auweraer, M., and De Schryver, F. C. The influence of solvent polarity on the nonradiative decay of exciplexes. Journal of the American Chemical Society 113, 6849-6857. 1991.

9. Swinnen, A. M., Van der Auweraer, M., De Schryver, F. C., Nakatami, K., Okada, T., and Mataga, N. Photophysics of the intramolecular exciplex formation in ω-(1-pyrenyl)-α-N,N-dimethylaminoalkanes. Journal of the American Chemical Society 109, 321-330. 1987.

10. Okada, T., Saito, T., Mataga, N., Sakata, Y., and Misumi, S. Solvent effects upon dynamic behaviour of intramolecular heteroexcimers. Bulletin of the Chemical Society of Japan 50(2), 331-336. 1977.

11. Okada, T., Fujita, T., Kubota, M., Masaki, S., and Mataga, N. Intramolecular electron donor-acceptor interactions in the excited state of (anthracene)-(CH₂)ₙ-(N,N-Dimethylaniline) systems. Chem.Phys.Lett. 14(5), 563-568. 1972.

12. Mataga, N. Photochemical Charge Transfer Phenomena-Picosecond Laser Photolysis Studies. Pure & Appl.Chem. 56(9), 1255-1268. 1984.

13. Verhoeven, J. W., Scherer, T., and Willemse, R. J. Solvent effects on the structure of fluorescent “exciplex” in rigidly-, flexibly-, and non-bridged donor-acceptor systems. Pure & Appl.Chem. 65(8), 1717-1722. 1993.

14. Verhoeven, J. W. Electron transport via saturated hydrocarbon bridges: ‘exciplex’ emission from flexible, rigit and semiflexible biochromophores. Pure & Appl.Chem. 62(8), 1585-1596. 1990.

15. Van der Auweraer, M., Viaene, L., Van Haver, Ph., and De Schryver, F. C. Influence of solvent polarity on the radiationless decay of the intramolecular exciplex of ω-phenyl-α-N,N-dimethylaminoalkanes. Journal of Physical Chemistry 97, 7178-7184. 1993.

16. Wegewijs, B., Hermant, R. M., Verhoeven, J. W., Kunst, A. G. M., and Rettschnick, R. P. H. Determination of the barrier to intramolecular exciplex formation in a jet-cooled, bichromophoric molecule. Chem.Phys.Lett. 140(6), 587-590. 1987.

17. Lewis, F. D and Cohen, B. E. Solvent-Dependent Behaviour of Phenanthrene-Amine Intramolecular Exciplexes. Journal of Physical Chemistry 98, 10591-10597. 1994.

18. Werner, U. Staerk H. Magnetic field effect in the recombination reaction of radical ion pairs: dependence on solvent dielectric constant. Journal of Physical Chemistry 99, 248-254. 1995.

19. Magnetic field effects on exciplex luminescence in water-tetrahydrofuran and water-dioxan mixtures. Journal of Physical Chemistry 101, 384-391. 1997.

20. Cox, G. S, Turro, N. J., Yang, N. C., and Chen, M. J. Intramolecular Exciplex Emission from Aqueous β-Cyclodextrin Solutions. Journal of the American Chemical Society 106,422-424. 1984.

21. Enhancement of intramolecular excimer formation of 1,3-bichromophoric propanes via application of high pressure and via complexation with cyclodextrins. Protection from oxygen quenching. Photochemistry and Photobiology 35, 325-329. 1982.

22. Benson, D. R and Fu, J. Exciplex Fluorescence in Inclusion Complexes of Naphtalene Derivatives. Tetrahedron Letters 37(28), 4833-4836. 2000. Ref Type: Journal (Full)

23. Pal, M. K. and Ghosh, J. K. Energy transfer and formation of exciplex between thiacyanine and acridine orange facilitated by anionic biopolymers and synthetic polymers. J.Photochem.Photobiol.A.Chem 78, 31-37. 1994. Ref Type: Journal (Full)

24. Ayala, N. P, Demas, J. N., and DeGraff, B. A. Exciplexes of (α-Diimine)ruthenium(II) Photosensitizers with silver(T). 2. Mixed water-acetonitrile solvent. Journal of Physical Chemistry 93, 4104-4109. 1989. Ref Type: Journal (Full)

25. Ayala, N. P, Demas, J. N., and DeGraff, B. A. Exciplexes of ruthenium(II) (α-diimine complexes with silver (1). Journal of the American Chemical Society 110, 1523-1529. 1988. Ref Type: Journal (Full)

26. (1982) CRC Handbook of Chemistry and Physics, 62 Ed., CRC Press, Inc.,

27. Reichardt, C. (1990) Solvent Effects in Organic Chemistry, VCH:Weinheim,

28. Wang, H., Zhang, B. W., and Cao, Y. Intramolecular charge transfer and exciplex formation in anthracene biochromophoric compounds. J.Photochem.Photobiol.A.Chem 92, 29-34. 1995. Ref Type: Journal (Full)

29. Bachmann, W. E. Syntheses of Phenanthrene Derivatives. I. Reactions of 9-Phenanthrylmagnesium Bromide. Journal of the American Chemical Society 56, 1363-1367. 1934. Ref Type: Journal (Full)

30. Muller, P. and Pautex, N. From Dichlorocyclopropanes to Furans and cycolpentadienes via Vinylcarbenes. Helv.Chim.Acta 71, 1630-1637. 1988. Ref Type: Journal (Full)

31. Bergmann, E. and Blum-Bergmann, O. Cyclopentenotriphenylene. Journal of the American Chemical Society 58, 1678-1681. 1936. Ref Type: Journal (Full)

32. Landini, D. and Rolla, F. A convenient synthesis of N-Alkylphthalimides in a solid-liquid two-phase system in the presence of phase-transfer catalysts. Synthesis, 389-390. 1976. Ref Type: Journal (Full)

101. Gordon, M. and Ware, W. R. (1975) The EXCIPLEX, Academic Press Inc., New York San Francisco London,

102. Birks, J. B. (1973) Organic Molecular Photophysics, John Wiley & Sons; London—New York—Toronto—Sydney,

103. Birks, J. B. (1973) Organic Molecular Photophysics, John Wiley & Sons; London—New York—Toronto—Sydney,

104. Masaki, S., Okada, T., Mataga, N., Sakata, Y., and Misumi, S. On the solvent-induced Changes of electronic structures of intramolecular exciplexes. Bulletin of the Chemical Society of Japan 49(5), 1277-1283. 1976.

105. Mataga, N. Photochemical Charge Transfer Phenomena-Picosecond Laser Photolysis Studies. Pure & Appl.Chem. 56(9), 1255-1268. 1984.

106. Van Haver, Ph., Helsen, N., Depaemelaere, S., Van der Auweraer, M., and De Schryver, F. C. The influence of solvent polarity on the nonradiative decay of exciplexes. Journal of the American Chemical Society 113, 6849-6857. 1991.

107. Swinnen, A. M., Van der Auweraer, M., De Schryver, F. C., Nakatami, K., Okada, T., and Mataga, N. Photophysics of the intramolecular exciplex formation in ω-(1-pyrenyl)-α-N,N-dimethylaminoalkanes. Journal of the American Chemical Society 109, 321-330.1987.

108. Okada, T., Saito, T., Mataga, N., Sakata, Y., and Misumi, S. Solvent effects upon dynamic behaviour of intramolecular heteroexcimers. Bulletin of the Chemical Society of Japan 50(2), 331-336. 1977.

109. Okada, T., Fujita, T., Kubota, M., Masaki, S., and Mataga, N. Intramolecular electron donor-acceptor interactions in the excited state of (anthracene)-(CH₂)ₙ-(N,N-Dimethylaniline) systems. Chem.Phys.Lett. 14(5), 563-568. 1972.

110. Verhoeven, J. W., Scherer, T., and Willemse, R. J. Solvent effects on the structure of fluorescent “exciplex” in rigidly-, flexibly-, and non-bridged donor-acceptor systems. Pure & Appl.Chem. 65(8), 1717-1722. 1993.

111. Verhoeven, J. W. Electron transport via saturated hydrocarbon bridges: ‘exciplex’ emission from flexible, rigit and semiflexible biochromophores. Pure & Appl.Chem. 62(8), 1585-1596. 1990.

112. Van der Auweraer, M., Viaene, L., Van Haver, Ph., and De Schryver, F. C. Influence of solvent polarity on the radiationless decay of the intramolecular exciplex of ω-phenyl-α-N,N-dimethylaminoalkanes. Journal of Physical Chemistry 97, 7178-7184. 1993.

113. On the electronic structure of exciplexes in α-(1- or 2-naphthyl)-ω-N,N-dialkylaminoalkanes. Journal of Photochemistry 28, 315-327. 1985.

114. Wegewijs, B., Hermant, R. M., Verhoeven, J. W., Kunst, A. G. M., and Rettschnick, R. P. H. Determination of the barrier to intramolecular exciplex formation in a jet-cooled, bichromophoric molecule. Chem.Phys.Lett. 140(6), 587-590. 1987.

115. Lewis, F. D and Cohen, B. E. Solvent-Dependent Behaviour of Phenanthrene-Amine Intramolecular Exciplexes. Journal of Physical Chemistry 98, 10591-10597. 1994.

116. Werner, U. Staerk H. Magnetic field effect in the recombination reaction of radical ion pairs: dependence on solvent dielectric constant. Journal of Physical Chemistry 99, 248-254. 1995.

117. Magnetic field effects on exciplex luminescence in water-tetrahydrofuran and water-dioxan mixtures. Journal of Physical Chemistry 101, 384-391. 1997.

118. Cox, G. S, Turro, N. J., Yang, N. C., and Chen, M. J. Intramolecular Exciplex Emission from Aqueous β-Cyclodextrin Solutions. Journal of the American Chemical Society 106, 422-424. 1984.

119. Enhancement of intramolecular excimer formation of 1,3-bichromophoric propanes via application of high pressure and via complexation with cyclodextrins. Protection from oxygen quenching. Photochemistry and Photobiology 35, 325-329. 1982.

120. Benson, D. R and Fu, J. Exciplex Fluorescence in Inclusion Complexes of Naphthalene Derivatives. Tetrahedron Letters 37(28), 4833-4836. 2000.

121. Pal, M. K. and Ghosh, J. K. Energy transfer and formation of exciplex between thiacyanine and acridine orange facilitated by anionic biopolymers and synthetic polymers. J.Photochem.Photobiol.A.Chem 78, 31-37. 1994.

122. (1982) CRC Handbook of Chemistry and Physics, 62 Ed., CRC Press, Inc.,

1. A compound capable of forming an intramolecular exciplex on photoirradiation of the compound in water, said compound comprising two exciplex forming partners, one being a donor moiety and the other an acceptor moiety, each having at least one aromatic nucleus and being connected by a saturated aliphatic chain having the flexibility to allow said partners to come into exciplex forming relationship.

2. A compound as claimed in claim 1 wherein the saturated aliphatic chain comprises 2 to 6 saturated atoms as the link between the two exciplex forming partners.

3. A compound as claimed in claim 2 wherein the linker comprises 2 to 4 saturated atoms as the link.

4. A compound as claimed in claim 3 wherein the linker comprises 3 saturated atoms as the link.

5. A compound as claimed in any one of claims 1 to 4 wherein the saturated atoms of the linker are comprised wholly or partly of carbon atoms.

6. A compound as claimed in claim 5 wherein the carbon atoms are provided by methylene groups.

7. A compound as claimed in claim 2 wherein the linker is —CH₂—CH₂— in which the free terminal buds are bonded to the exciplex forming partners.

8. A compound as claimed in claim 4 wherein the linker is —CH₂—CH₂—CH₂— or —CH₂—NH—CH₂— in which the free terminal bonds are bonded to the exciplex forming partners.

9. A compound as claimed in any one of claims 1 to 8 wherein the aromatic nucleic of the donor and acceptor moieties are selected from
(i) benzene, naphthalene, anthracene, phenanthrene, pyrene and chrysene, phenanthrene;
(ii) derivatives of residues of compounds identified under (i); and
(iii) phthalimide or substituted derivatives thereof

10. A compound as claimed in claim 9 wherein the donor moiety is a 1-pyrenyl group and the acceptor moiety is an amino, N-alkylamino, N,N-dialkylamino substituted benzene or naphthalene nucleus.

11. A compound as claimed in claim 10 wherein amino, N-alkylamino or N,N-dialkylamino group is at the 4-position at which the linker is bonded to the benzene or naphthalene nucleus.

12. A compound as claimed in claim 10 or 11 wherein the acceptor moiety is an N-alkylamino or N,N-dialkylamino substituted benzene or naphthalene nucleus and the alkyl groups have one to two carbon atoms.

13. The compound

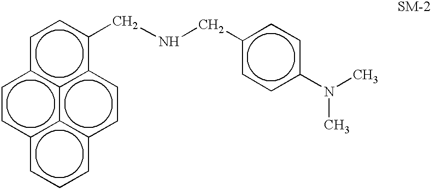

14. The compound

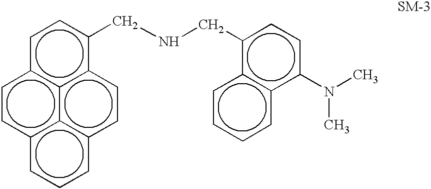

15. The compound

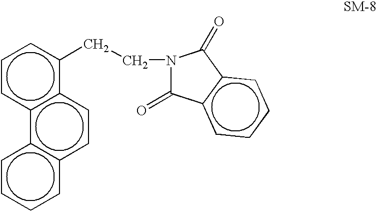

16. A compound as claimed in any one of claims 1 to 12 wherein the linker is substituted with a functional group permitting covalent attachment of the compound as a label to a molecule or other entity to be labelled.

17. A compound as claimed in claim 16 wherein the functional group is an amino group.

18. A molecule or other entity labelled with a compound as claimed in any one of claims 1 to 17.

19. An entity as claimed in claim 18 which is a labelled nucleic acid.

20. An entity as claimed in claim 18 which is labelled oligonucleotide.

21. An entity as claimed in claim 18 which is a labelled protein.

22. A entity as claimed in claim 18 which is a labelled ceramic, transistor, semiconductor, insulator or glass.

23. A method of generating an exciplex comprising irradiating a compound as claimed in any one of claims 1 to 17 or entity as claimed in any one of claims 19 to 22 with electromagnetic radiation of a wavelength appropriate for exciplex formation by said donor and acceptor moieties.

24. A method as claimed in claim 23 additionally comprising the step of detecting for exciplex formation.

25. A method of detection wherein there is used as a label a compound as claimed in any one of claims 1 to 17 and the method comprises irradiating a sample under test with electromagnetic radiation capable of providing exciplex formation in said label if present and detecting for exciplex formation.

26. A method as claimed in any one of claims 23 to 25 which is effected in a polar medium.

27. A method as claimed in claim 14 wherein the polar medium is an aqueous medium.

28. A method as claimed in claim 26 or 27 wherein the exciplex emission is pH sensitive.

29. A method as claimed in claim 28 which involves adjusting pH to obtain or modify exciplex emission.

30. A method as claimed in claim 28 which is effected to 30. A method as claimed in claim 28 which is effected to
(a) register pH changes in an environment such as a living or fixed cell, or in a curvette or other container including cell sorter devices, and be used in assays to other determinations, or
(b) induce a signal by manipulation of the pH of the environment externally, such as by added acid, base or other change in conditions which leads to a pH change.
